(19) United States
(12) Patent Application Publication
(10) Pub. No.: US 2022/0378321 A1
(43) Pub. Date: Dec. 1, 2022

(54) METHODS AND SYSTEMS FOR ASSESSING SEVERITY OF RESPIRATORY DISTRESS OF A PATIENT

(71) Applicant: SOCOVAR S.E.C., Montreal (CA)

(72) Inventors: Haythem REHOUMA, Lachine (CA); Rita NOUMEIR, Saint-Laurent (CA); Philippe JOUVET, Montreal (CA); Sandrine ESSOURI, Montreal (CA)

(21) Appl. No.: 17/763,319

(22) PCT Filed: Sep. 23, 2020
(86) PCT No.: PCT/CA2020/051273

§ 371 Date: Mar. 24, 2022

Related U.S. Application Data
(60) Provisional application No. 62/904,980, filed on Sep. 24, 2019.

(51) Int. Cl. A61B 5/08; A61B 5/00; A61B 5/11; A61B 5/113

(52) U.S. Cl. CPC A61B 5/0816; A61B 5/0077; A61B 5/1114; A61B 5/1135

(57) ABSTRACT

There is described a method of assessing severity of a respiratory distress of a patient. The method generally has, using a three dimensional (3D) camera, generating at least a 3D image encompassing at least a thorax region and an abdomen region of the patient; and using a computer, accessing said 3D image; identifying thorax coordinates indicating coordinates of at least a point of the thorax region of the patient in the 3D image; identifying abdomen coordinates indicating coordinates of at least a point of the abdomen region of the patient in the 3D image; determining a thoraco-abdominal distance based on the thorax coordinates and on the abdomen coordinates; comparing the thoraco-abdominal distance with a threshold; and generating a signal based on said comparison, said signal being indicative of a degree of severity of the respiratory distress of the patient.

### FIELD

The improvements generally relate to respiratory distress and more particularly relate to assessing respiratory distress of a patient.

### BACKGROUND

Assessing respiratory distress of a patient generally requires highly trained healthcare professionals to be present near the patient. Even when such healthcare professionals are examining the patient, noticing subtle signs of respiratory distress, including retraction signs in the upper body region of the patient and/or thoraco-abdominal asynchrony, can remain challenging. There thus remains room for improvement.

### SUMMARY

It was found that there is a need in the medical industry for methods and systems which can evaluator and monitor key respiratory patterns and indicators of a breathing patient without the need for healthcare professional(s).

In accordance with a first aspect of the present disclosure, there is provided a method of assessing severity of a respiratory distress of a patient, the method comprising: using a three dimensional (3D) camera, generating at least a 3D image encompassing at least a thoraco-abdominal region of said patient at a given moment in time; and using a computer, accessing said 3D image; identifying first coordinates indicating coordinates of at least a first point of said thoraco-abdominal region of said patient in said 3D image; identifying second coordinates indicating coordinates of at least a second point of said thoraco-abdominal region of said patient in said 3D image; determining a distance based on said first and second coordinates; comparing said distance with a threshold; and generating a signal based on said comparison, said signal being indicative of a degree of severity of said respiratory distress of said patient.

Further in accordance with the first aspect of the present disclosure, said thoraco-abdominal region can for example have at least a thorax region and an abdominal region, said first point can for example be associated with said thorax region of said patient in said 3D image, said second point can for example be associated with said abdominal region of said patient, and said distance can for example correspond to a thoraco-abdominal distance indicating a distance between said thorax region and said abdominal region of said patient.

Still further in accordance with the first aspect of the present disclosure, said thoraco-abdominal region can for example have at least a secondary respiratory muscle and an anatomical landmark, said first point can for example be associated with said secondary respiratory muscle of said patient in said 3D image, and said second point can for example be associated with said anatomical landmark of said patient in said 3D image.

Still further in accordance with the first aspect of the present disclosure, said secondary respiratory muscle can for example be selected among a group consisting of: a sternocleidomastoid muscle, a scalene muscle, and an intercostal muscle.

Still further in accordance with the first aspect of the present disclosure, said anatomical landmark can for example be selected among a group consisting of: a region around a clavicle of said patient, a region below a neck of said patient and a region between ribs of said patient.

Still further in accordance with the first aspect of the present disclosure, the method can for example further comprise generating an alert when said distance exceeds said threshold.

Still further in accordance with the first aspect of the present disclosure, said moment in time can for example correspond to at least one of an end of an inspiration and an end of an expiration of said patient.

Still further in accordance with the first aspect of the present disclosure, the method further can for example comprise repeating said method a given number of times thereby monitoring said distance over time.

Still further in accordance with the first aspect of the present disclosure, the method can for example further comprise displaying said monitored distance on a display screen.

Still further in accordance with the first aspect of the present disclosure, wherein said 3D image can for example be provided in the form of a cloud of points.

In accordance with a second aspect of the present disclosure, there is provided a system for assessing severity of a respiratory distress of a patient, the system comprising: a three dimensional (3D) camera generating at least a 3D image encompassing at least a thoraco-abdominal region of said patient at a given moment in time; and a computer being communicatively coupled to said 3D camera, said computer having a processor and a memory having stored thereon instructions that when executed by said processor perform the steps of: accessing said 3D image; identifying first coordinates indicating coordinates of at least a first point of said thoraco-abdominal region of said patient in said 3D image; identifying second coordinates indicating coordinates of at least a second point of said thoraco-abdominal region of said patient in said 3D image; determining a distance based on said first and second coordinates; comparing said distance with a threshold; and generating a signal based on said comparison, said signal being indicative of a degree of severity of said respiratory distress of said patient.

Further in accordance with the second aspect of the present disclosure, said thoraco-abdominal region can for example have at least a thorax region and an abdominal region, said first point can for example be associated with said thorax region of said patient in said 3D image, said second point can for example be associated with said abdominal region of said patient, and said distance can for example correspond to a thoraco-abdominal distance storable on said memory.

Still further in accordance with the second aspect of the present disclosure, said thoraco-abdominal region can for example have at least a secondary respiratory muscle and an anatomical landmark, said first point can for example be associated with said secondary respiratory muscle of said patient in said 3D image, and said second point can for example be associated with said anatomical landmark of said patient in said 3D image.

Still further in accordance with the second aspect of the present disclosure, said secondary respiratory muscle can for example be selected among a group consisting of: a sternocleidomastoid muscle, a scalene muscle, and an intercostal muscle.

Still further in accordance with the second aspect of the present disclosure, said anatomical landmark can for example be selected among a group consisting of: a region around a clavicle of said patient, a region below a neck of said patient and a region between ribs of said patient.

Still further in accordance with the second aspect of the present disclosure, the system can for example further comprise an indicator generating an alert when said distance exceeds said threshold.

Still further in accordance with the second aspect of the present disclosure, said moment in time can for example correspond to at least one of an end of an inspiration and an end of an expiration of said patient.

Still further in accordance with the second aspect of the present disclosure, the system can for example further comprise repeating said 3D camera generates a plurality of 3D images as said patient breathes, said instructions being performed for at least some of said 3D images thereby monitoring said distance over time.

Still further in accordance with the second aspect of the present disclosure, the system can for example further comprise a display screen displaying said monitored distance.

In accordance with a third aspect of the present disclosure, there is provided a method of assessing severity of a respiratory distress of a patient, the method comprising: using a three dimensional (3D) camera, generating a plurality of 3D images encompassing at least a thoraco-abdominal region of said patient at a plurality of moments in time; and using a computer, accessing said plurality of 3D images; identifying a plurality of thoraco-abdominal coordinates indicating coordinates of at least a point of said thoraco-abdominal region of said patient in said plurality of 3D images; determining a direction of movement of said thoraco-abdominal region across said moments in time based on said identified thoraco-abdominal coordinates; upon determining that said direction of movement switches from a first direction of movement to a second direction of movement opposite to said first direction of movement, identifying at least one of a first 3D image of said plurality of 3D images corresponding to an end of an inspiration of said patient and a second 3D image of said plurality of 3D images corresponding to an end of an expiration of said patient; and generating a signal based on at least one of said first and second 3D images, said signal being indicative of a degree of severity of said respiratory distress of said patient.

Further in accordance with the third aspect of the present disclosure, said computer can for example further identify first coordinates indicating coordinates of at least a first point of said thoraco-abdominal region of said patient in at least one of said first and second 3D images, identifying second coordinates indicating coordinates of at least a second point of said thoraco-abdominal region of said patient in at least one of said first and second 3D images, determining a distance based on said first and second coordinates in at least one of said first and second 3D images, and comparing said distance with a threshold, said signal being based on said comparison.

Still further in accordance with the third aspect of the present disclosure, said thoraco-abdominal region can for example have at least a thorax region and an abdominal region, said first point can for example be associated with said thorax region of said patient in said 3D image, said second point can for example be associated with said abdominal region of said patient, and said distance corresponding to a thoraco-abdominal distance.

Still further in accordance with the third aspect of the present disclosure, said point of said thoraco-abdominal region can for example correspond to a first point of a thorax region of said patient, said direction of movement being a first direction of movement, said computer can for example further identify abdominal coordinates indicating coordinates of at least a second point of said abdominal region of said patient in said plurality of 3D images, determining a second direction of movement of said abdominal region across said moments in time based on said identified abdominal coordinates, and comparing said first and second directions of movement to one another, said signal being based on said comparison.

Still further in accordance with the third aspect of the present disclosure, the method can for example further comprise generating an alert when said first and second directions of movement are opposite to one another.

Still further in accordance with the third aspect of the present disclosure, the method can for example further comprise repeating said method a given number of times thereby monitoring thoraco-abdominal asynchrony of said patient over time.

Still further in accordance with the third aspect of the present disclosure, the method can for example further comprise, based on said first and second 3D images, determining a retraction distance corresponding to a distance between coordinates of said point of said thoraco-abdominal region in said first 3D image and coordinates of said point of said thoraco-abdominal region in said second 3D image.

Still further in accordance with the third aspect of the present disclosure, the method can for example further comprise generating an alert when said retraction distance exceeds a given threshold.

Still further in accordance with the third aspect of the present disclosure, the method can for example further comprise determining a tidal volume corresponding to a volume extending between a surface of said thoraco-abdominal region in said first 3D image and a surface of said thoraco-abdominal region in said second 3D image.

Still further in accordance with the third aspect of the present disclosure, said determining said direction of movement can for example include monitoring a curvature value associated with said point across said plurality of 3D images.

In accordance with a fourth aspect of the present disclosure, there is provided a system for assessing severity of a respiratory distress of a patient, the system comprising: a three dimensional (3D) camera generating a plurality of 3D images encompassing at least a thoraco-abdominal region of said patient at a plurality of moments in time; and a computer being communicatively coupled to said 3D camera, said computer having a processor and a memory having stored thereon instructions that when executed by said processor perform the steps of: accessing said plurality of 3D images; identifying a plurality of thoraco-abdominal coordinates indicating coordinates of at least a point of said thoraco-abdominal region of said patient in said plurality of 3D images; determining a direction of movement of said thoraco-abdominal region across said moments in time based on said identified thoraco-abdominal coordinates; upon determining that said direction of movement switches from a first direction of movement to a second direction of movement opposite to said first direction of movement, identifying at least one of a first 3D image of said plurality of 3D images corresponding to an end of an inspiration of said patient and a second 3D image of said plurality of 3D images corresponding to an end of an expiration of said patient; and generating a signal based on at least one of said first and second 3D images, said signal being indicative of a degree of severity of said respiratory distress of said patient.

Further in accordance with the fourth aspect of the present disclosure, said computer can for example further identify first coordinates indicating coordinates of at least a first point of said thoraco-abdominal region of said patient in at least one of said first and second 3D images, identifying second coordinates indicating coordinates of at least a second point of said thoraco-abdominal region of said patient in at least one of said first and second 3D images, determining a distance based on said first and second coordinates in at least one of said first and second 3D images, and comparing said distance with a threshold, said signal being based on said comparison.

Still further in accordance with the fourth aspect of the present disclosure, said thoraco-abdominal region can for example have at least a thorax region and an abdominal region, said first point being associated with said thorax region of said patient in said 3D image, said second point being associated with said abdominal region of said patient, and said distance corresponding to a thoraco-abdominal distance.

Still further in accordance with the fourth aspect of the present disclosure, said point of said thoraco-abdominal region can for example correspond to a first point of a thorax region of said patient, said direction of movement being a first direction of movement, said computer further identifying abdominal coordinates indicating coordinates of at least a second point of said abdominal region of said patient in said plurality of 3D images, determining a second direction of movement of said abdominal region across said moments in time based on said identified abdominal coordinates, and comparing said first and second directions of movement to one another, said signal being based on said comparison.

Still further in accordance with the fourth aspect of the present disclosure, the system can for example further comprise generating an alert when said first and second directions of movement are opposite to one another.

Still further in accordance with the fourth aspect of the present disclosure, the system can for example further comprise repeating said steps a given number of times thereby monitoring thoraco-abdominal asynchrony over time.

Still further in accordance with the fourth aspect of the present disclosure, the system can for example further comprise, based on said first and second 3D images, determining a retraction distance corresponding to a distance between coordinates of said point of said thoraco-abdominal region in said first 3D image and coordinates of said point of said thoraco-abdominal region in said second 3D image.

Still further in accordance with the fourth aspect of the present disclosure, the system can for example further comprise an indicator generating an alert when said retraction distance exceeds a given threshold.

Still further in accordance with the fourth aspect of the present disclosure, the system can for example further comprise determining a tidal volume corresponding to a volume extending between a surface of said thoraco-abdominal region in said first 3D image and a surface of said thoraco-abdominal region in said second 3D image.

Still further in accordance with the fourth aspect of the present disclosure, said determining said direction of movement can for example include monitoring a curvature value associated with said point across said plurality of 3D images.

In accordance with a fifth aspect of the present disclosure, there is provided a method of evaluating a respiratory parameter of a breathing patient, the method comprising: using a three dimensional (3D) camera, generating a plurality of 3D images encompassing at least a thoraco-abdominal region of said patient at a plurality of moments in time as said patient breathes; and using a computer, accessing said plurality of 3D images; processing at least some of said 3D images; evaluating a respiratory parameter based on said processing; and generating a signal based on said respiratory parameter.

Further in accordance with the fifth aspect of the present disclosure, said evaluating can for example include determining a tidal volume corresponding to a volume extending between a surface of said thoraco-abdominal region in a first 3D image of said 3D images corresponding to an end of an inspiration of said patient and a surface of said thoraco-abdominal region in a second 3D image of said 3D images corresponding to an end of an expiration of said patient.

Still further in accordance with the fifth aspect of the present disclosure, said evaluating can for example include determining a respiratory rate of said patient.

Still further in accordance with the fifth aspect of the present disclosure, said determining said respiratory rate can for example include evaluating a rate at which a point of said thoraco-abdominal region oscillates in a back and forth manner across the plurality of 3D images.

Still further in accordance with the fifth aspect of the present disclosure, said evaluating can for example include determining a retraction distance corresponding to a distance between a surface of said thoraco-abdominal region in a first 3D image corresponding to an end of an inspiration of said patient and a surface of said thoraco-abdominal region in a second 3D image corresponding to an end of an expiration of said patient.

Still further in accordance with the fifth aspect of the present disclosure, the method can for example further comprise monitoring said respiratory parameter over time.

Still further in accordance with the fifth aspect of the present disclosure, the method can for example further comprise generating an alert upon determining said monitored respiratory parameter exceeds a given threshold.

Still further in accordance with the fifth aspect of the present disclosure, the method can for example further comprise displaying said alert on a display screen.

In accordance with a sixth aspect of the present disclosure, there is provided a system for evaluating a respiratory parameter of a breathing patient, the system comprising: using a three dimensional (3D) camera, generating a plurality of 3D images encompassing at least a thoraco-abdominal region of said patient at a plurality of moments in time as said patient breathes; and using a computer, accessing said plurality of 3D images; processing at least some of said 3D images; evaluating a respiratory parameter based on said processing; generating a signal based on said respiratory parameter.

Further in accordance with the sixth aspect of the present disclosure, said evaluating can for example include determining a tidal volume corresponding to a volume extending between a surface of said thoraco-abdominal region in a first 3D image corresponding to an end of an inspiration of said patient and a surface of said thoraco-abdominal region in a second 3D image corresponding to an end of an expiration of said patient.

Still further in accordance with the sixth aspect of the present disclosure, said evaluating can for example include determining a respiratory rate of said patient.

Still further in accordance with the sixth aspect of the present disclosure, said determining said respiratory rate includes evaluating the rate at which a point of said thoraco-abdominal region oscillates across the plurality of 3D images.

Still further in accordance with the sixth aspect of the present disclosure, said evaluating can for example include determining a retraction distance corresponding to a distance between a surface of said thoraco-abdominal region in a first 3D image corresponding to an end of an inspiration of said patient and a surface of said thoraco-abdominal region in a second 3D image corresponding to an end of an expiration of said patient.

Still further in accordance with the sixth aspect of the present disclosure, the system can for example further comprise monitoring said respiratory parameter over time.

Still further in accordance with the sixth aspect of the present disclosure, the system can for example further comprise generating an alert upon determining said monitored respiratory parameter exceeds a given threshold.

Still further in accordance with the sixth aspect of the present disclosure, the system can for example further comprise displaying said alert on a display screen.

Many further features and combinations thereof concerning the present improvements will appear to those skilled in the art following a reading of the instant disclosure.

### DESCRIPTION OF THE FIGURES

In the figures,

FIG. 1 is a schematic view of a first example of a system for assessing severity of a respiratory distress of a patient, including a 3D camera and a computer, in accordance with one or more embodiments;

FIG. 1A is a graph showing an example of a 3D image of the patient of FIG. 1, in accordance with one or more embodiments;

FIG. 2 is a schematic view of an example of a computing device of the computer of FIG. 1, in accordance with one or more embodiments;

FIG. 3 is a flow chart of a first example of a method for assessing severity of a respiratory distress of a patient using the system of FIG. 1, in accordance with one or more embodiments;

FIG. 4 is a schematic view of a second example of a system for assessing severity of a respiratory distress of a patient, including a 3D camera and a computer, in accordance with one or more embodiments;

FIG. 4A is a graph showing an example of a 3D image of the patient of FIG. 4, in accordance with one or more embodiments;

FIG. 4B is a graph showing an example of a subsequent 3D image of the patient of FIG. 4, in accordance with one or more embodiments;

FIG. 5 is a flow chart of a second example of a method for assessing severity of a respiratory distress of a patient using the system of FIG. 4, in accordance with one or more embodiments;

FIG. 6 is an image of an example of a stereo camera of type Kinect v2, in accordance with one or more embodiments;

FIG. 7 is a flow chart of a method of calculating a volume of an upper body portion of a patient, in accordance with one or more embodiments;

FIGS. 8A-F include camera placement examples, in which the cameras are placed at the bed top in FIG. 8A, at the bed bottom in FIG. 8B, at the top right and bottom left in FIG. 8C, at top left and bottom right in FIG. 8D, at bed right side in FIG. 8E, at bed left side in FIG. 8F, in accordance with one or more embodiments;

FIG. 9 is a schematic view showing corresponding pairs of 3D points between surfaces before and after respiratory displacement of the test lung surface, in accordance with one or more embodiments;

FIG. 10 are schematic views showing steps of a method of assessing respiratory distress of a patient, in accordance with one or more embodiments;

FIG. 11 show a schematic visualization of the proposed camera setup and their resulting views in insets A and B showing a baby mannequin, in accordance with one or more embodiments;

FIG. 12 are graphs showing volume variation of a patient as determined with a method of calculating a volume of an upper body portion of a patient, in accordance with one or more embodiments;

FIG. 13 is a schematic view showing an exemplary motion extraction technique based on comparing distances from a RGB-D sensor, whose center is the origin of the coordinate system, in accordance with one or more embodiments;

FIG. 14 is a schematic view of a system for assessing respiratory distress of a patient, in accordance with one or more embodiments;

FIG. 15 is a schematic view of a cloud to sensor distance estimation at the frame j, in accordance with one or more embodiments;

FIG. 16 include regions extraction obtained for 3D images in the tested sequences with the first three 3D images representing normal inspiration, the following three 3D images representing normal expiration, and the remaining 3D images representing TAA, in accordance with one or more embodiments;

FIG. 17 is a schematic view showing computing of cloud-to-cloud maximal displacement between surfaces, in accordance with one or more embodiments;

FIG. 18 show graphs of distance as a function of time for different types of respirations: normal respiration, mild TAA, severe TAA and irregular mode, in accordance with one or more embodiments;

FIG. 19 is a flow chart of another example method of assessing respiratory distress of a patient, showing a step of mean curvature determination, in accordance with one or more embodiments;

FIGS. 20A and 20B are schematic views of oscillating circles adjoining corresponding curves, in accordance with one or more embodiments;

FIGS. 21A, 21B and 21C are schematic views of curved surfaces, showing respective mean curvatures thereof, in accordance with one or more embodiments;

FIGS. 22A and 22B are flowcharts of another example method of assessing respiratory distress of a patient, showing curvature computation and comparison, in accordance with one or more embodiments;

FIGS. 23A and 23B are schematic views showing curves of increasing and decreasing curvatures, respectively, in accordance with one or more embodiments; and

FIGS. 24A and 24B are schematic views of curves associated to thorax and abdomen regions as they are modified during a respiration cycle, in accordance with one or more embodiments.

### DETAILED DESCRIPTION

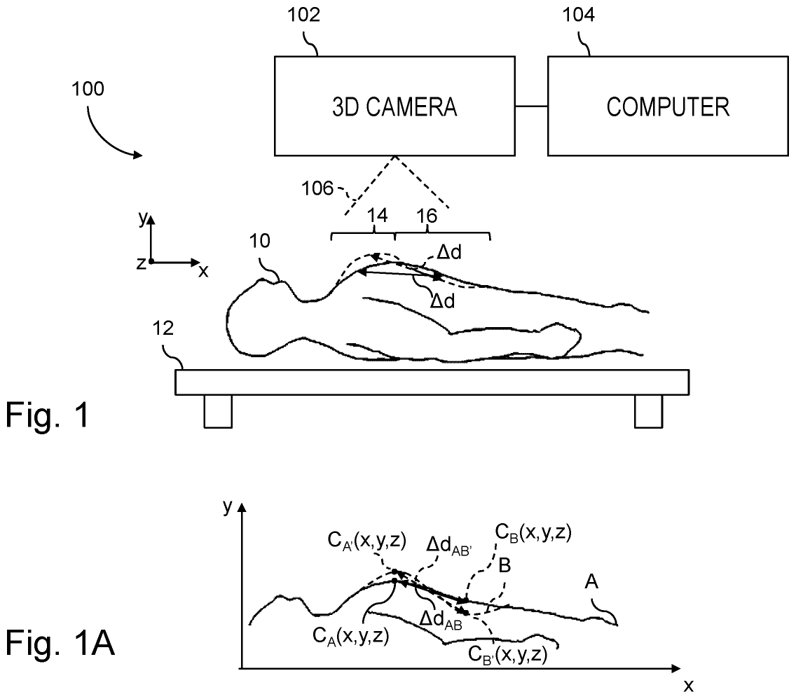

FIG. 1 shows an example of a system 100 for assessing severity of a respiratory distress of a patient 10. In this embodiment, the system 100 can be positioned proximate a hospital bed 12 on which the patient 10 lies. As depicted, the system 100 has a 3D camera 102 and a computer 104 which is communicatively coupled to the 3D camera 102. The communication between the 3D camera 102 and the computer 104 can be wired, wireless, or a combination of both depending on the embodiment.

As shown, the 3D camera 102 has a field of view 106 encompassing at least a thoraco-abdominal region of the patient, including a thorax region 14 and an abdomen region 16 of the patient 10. As such, the 3D camera 102 is used to generate one or more 3D images of the patient 10, and more particularly of the thorax and abdomen regions 14 and 16 of the patient 10. The 3D camera 102 can be provided in the form of a stereo camera, a structured-light 3D scanner, a movable laser range finder, an array of range finders, a time-of-flight camera, and or any other suitable type of 3D camera. The 3D image can include, but not limited to, a cloud of points having respective coordinates in an arbitrary reference system (x,y,z). FIG. 1A shows first and second clouds of points A and B as generated by the 3D camera 102. As depicted, the first cloud of points A represents the thorax and abdomen regions 14 and 16 after an expiration of the patient 10 and the second cloud of points B represents the thorax and abdomen regions 14 and 16 during an inspiration of the patient 10. As the clouds of points A and B are shown to extend only in the x-y plane in this example, the clouds of points can extend in the three-dimensional reference system (x,y,z). Such 3D images can be generated at a given frequency as the patient 10 is under observation. For example, the frequency at which 3D images are generated can vary between 1 Hz and 50 Hz, and is most preferably about 30 Hz.

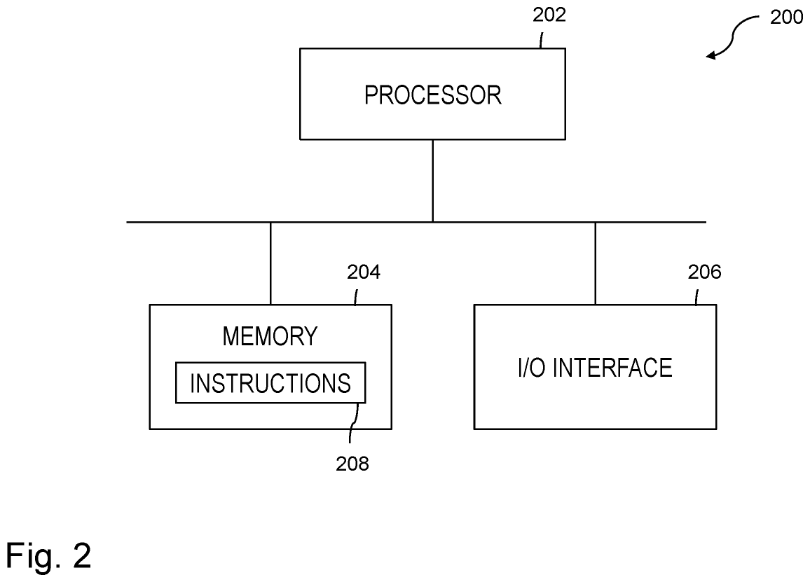

The computer 104 can be provided as a combination of hardware and software components. The hardware components can be implemented in the form of a computing device 200, an example of which is described with reference to FIG. 2.

Referring to FIG. 2, the computing device 200 can have a processor 202, a memory 204, and I/O interface 206. Instructions 208 for assessing severity of a respiratory distress of the patient 10 can be stored on the memory 204 and accessible by the processor 202.

The processor 202 can be, for example, a general-purpose microprocessor or microcontroller, a digital signal processing (DSP) processor, an integrated circuit, a field programmable gate array (FPGA), a reconfigurable processor, a programmable read-only memory (PROM), or any combination thereof.

The memory 204 can include a suitable combination of any type of computer-readable memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 206 enables the computing device 200 to interconnect with one or more input devices, such as mouse(s), keyboard(s), button(s), 3D camera(s) and the like, or with one or more output devices such as network(s), database(s), display(s), remote network(s) and the like.

Each I/O interface 206 enables the computer 104 to communicate with other components, to exchange data with other components, to access and connect to network resources, to server applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

The computer 104 can be configured to implement software application(s) that is(are) configured to receive signal(s) and/or data being indicative of the instructions 208 and to determine the instructions 208 upon processing the signal(s) and/or data. In some embodiments, the software application(s) is(are) stored on the memory 204 and accessible by the processor 202 of the computing device 200.

The computing device 200 and the software application(s) described above are meant to be examples only. Other suitable embodiments of the computer 104 can also be provided, as it will be apparent to the skilled reader.

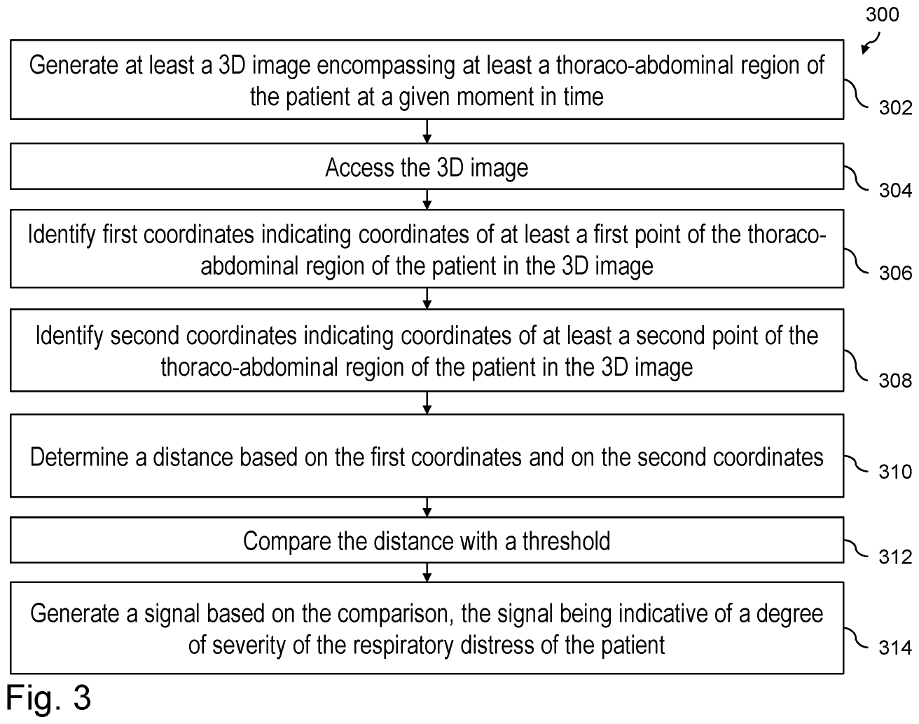

Referring now to FIG. 3, there is shown an example of a method 300 of assessing severity of a respiratory distress of the patient 10. The method 300 will be described with reference to FIGS. 1 and 1A for ease of reading.

As shown, at step 302, the 3D camera 102 generates a 3D image encompassing at least the thoraco-abdominal region of the patient, and more specifically the thorax region 14 and the abdomen region 16 of the patient 10 in this example. The 3D image can be stored on the memory 204, or stored on a remote memory as desired. The 3D image can also be communicated to a remote network for further processing and/or storing.

At step 304, the computer 104 accesses the 3D image. The computer 104 can access the 3D image by accessing its own memory or a remote memory and/or by communicating with the network depending on the embodiment.

At step 306, the computer 104 identifies first coordinates indicating coordinates of at least a first point of the thoraco-abdominal region of the patient 10 in the 3D image. In some embodiments, the first point can be associated with the thorax region 14 of the patient. In these embodiments, the first coordinates are referred to as thorax coordinates.

At step 308, the computer 104 identifies second coordinates indicating coordinates of at least a different, second point of the thoraco-abdominal region of the patient 10 in the 3D image. In some embodiments, the second point can be associated with the abdominal region 16 of the patient. In such embodiments, the second coordinates are abdominal coordinates.

At step 310, the computer 104 determines a distance based on the first and second coordinates. For instance, in embodiments where the first and second coordinates correspond to thorax and abdominal coordinates, respectively, the determined distance can correspond to a thoraco-abdominal distance. In some embodiments, the distance is determined using basic linear algebra calculations, and more specifically is defined as the shortest distance between the first and second points, e.g., the shortest distance between the thorax and abdomen regions 14 and 16. The distance can be the Euclidian distance, the L1 norm and any other distance. It is noted that the distance is preferably estimated at the end of inspiration in the thoraco-abdominal respiratory movement of the patient. For instance, a generally accepted definition of the thoraco-abdominal asynchrony (TAA) submits that it is the paradoxical motion (PM) of the chest and abdomen, during which the abdomen moves outward while the chest moves inward during inspiration. Such an asynchrony may be better appreciated at the end of the inspiration. Accordingly, in some embodiments, the moment in time at which the 3D image is generated at step 302 may correspond to the end of the inspiration of the patient. However, in some other embodiments, the 3D image can be generated as the patient 10 expires or inspires, or at the end of an expiration.

At step 312, the computer 104 compares the distance determined at step 310 with a threshold. The threshold can be stored on an accessible or network. In some embodiments, the threshold can be modified on the go via one or more user inputs, taking consideration for example of the dimensions of the patient 10. Numerical values for this threshold are patient-dependent. Accordingly, reference values for the threshold could be obtained for different types of patients (e.g., male, female, adult, kid, elderly).

At step 314, the computer 104 generates a signal based on the comparison performed at step 312, in which the so-generated signal is indicative of a degree of severity of the respiratory distress of the patient. For example, the degree of severity of the respiratory distress can be more severe upon determining that the distance is greater than the threshold. The degree of severity can be less severe upon determining that the distance is below the threshold.

It is intended that in some embodiments the method 300 can include a step of generating an alert when the distance exceeds the threshold. The alert may be displayed on a display screen in some embodiments. The alert may be auditory in some alternate embodiments. Additionally, or alternately, the method 300 can be repeated a number of times to monitor the distance over time. For example, monitoring the thoraco-abdominal distance as a patient breathes can help to detect respiratory distress as it occurs.

Referring back to FIG. 1A, there is shown a solid line representing a 3D image of a patient 10 at a first moment in time and a dashed line representing a 3D image of the patient 10 at a second moment in time. In this example, the solid line corresponds to the first cloud of points A whereas the dashed line corresponds to the second cloud of points B.

As shown, using the 3D image of the patient 10 at the first moment in time, the computer 104 identifies the thorax coordinates C_{A}(x,y,z) in the 3D image, in which the thorax coordinates C_{A}(x,y,z) indicate coordinates of at least a point C_{A} of the thorax region 14 of the patient 10 in the 3D image. Abdomen coordinates C_{B}(x,y,z) in the 3D image indicating coordinates of at least a point C_{B} of the abdomen region 16 of the patient 10 in the 3D image are also identified. The computer 104 then determines a thoraco-abdominal distance Δd_{AB} based on the thorax coordinates C_{A}(x,y,z) and on the abdomen coordinates C_{B}(x,y,z). As discussed, in this embodiment, the computer 104 performs a comparison between the thoraco-abdominal distance Δd_{AB} and a threshold Δdₜₕᵣₑₛ It is intended that, based on the comparison, the computer 104 generates a signal which is indicative of a degree of severity of the respiratory distress of the patient. For instance, in this case, the thoraco-abdominal distance Δd_{AB} is below the threshold Δdₜₕᵣₑₛ and accordingly the so-generated signal can be indicative of a low degree of severity of the respiratory distress of the patient.

In contrast, using the 3D image of the patient 10 at the second moment in time, the computer 104 identifies the thorax coordinates C_{A′}(x,y,z) in the 3D image, in which the thorax coordinates C_{A′}(x,y,z) indicate coordinates of at least a point C_{A′} of the thorax region 14 of the patient 10 in the 3D image. Also, the computer 104 identifies the abdomen coordinates C_{B′}(x,y,z) in the 3D image, in which the abdomen coordinates C_{B′}(x,y,z) indicate coordinates of at least a point C_{B′} of the abdomen region 16 of the patient 10 in the 3D image. The computer then determines a thoraco-abdominal distance Δd_{AB}′ based on the thorax coordinates C_{A′} (x,y,z) and on the abdomen coordinates C_{B′} (x,y,z). The thoraco-abdominal distance Δd_{AB}′ and a threshold Δdₜₕᵣₑₛ are then compared by the computer 104. It is intended that the computer 104 then generates a signal based on the comparison, which signal is indicative of a degree of severity of the respiratory distress of the patient. In this specific case, the thoraco-abdominal distance Δd_{AB} exceeds the threshold Δdₜₕᵣₑₛ and accordingly the signal is indicative of a high degree of severity of the respiratory distress of the patient.

It is thus intended that the relative difference between the thoraco-abdominal distance and the threshold can be indicative of the degree of severity of the respiratory of the patient. In some embodiments, the degree of severity can be expressed as a quantitative value, e.g., a value of a scale of 1 to 3. For instance, the value may be 1 whenever the thoraco-abdominal distance is below the threshold Δdₜₕᵣₑₛ, the value may be 2 when the thoraco-abdominal distance Δd generally corresponds to the threshold Δdₜₕᵣₑₛ and the value may be 3 when the thoraco-abdominal distance Δd is below the threshold Δdₜₕᵣₑₛ In some other embodiments, the degree of severity can be expressed in the form of a percentage relative to the threshold, in the form of a value between 1 and 100, and the like.

In some embodiments, the first point discussed at step 306 is associated with a secondary respiratory muscle of the patient in the 3D image whereas the second point discussed at step 308 is associated with an anatomical landmark of the patient in the 3D image. Accordingly, the distance to be determined at step 310 may not be a thoraco-abdominal distance, but rather another type of distance useful in determining respiratory distress of the patient, if any. An example of such a distance includes, but is not limited to, an intercostal retraction distance. In these embodiments, the secondary respiratory muscle can be selected among a group consisting of: a sternocleidomastoid muscle, a scalene muscle, and an intercostal muscle. Moreover, in these embodiments, the anatomical landmark can be selected among a group consisting of: a region around a clavicle of the patient, a region below a neck of the patient and a region between ribs of the patient. Other respiratory useful distance may be determined in some other embodiments.

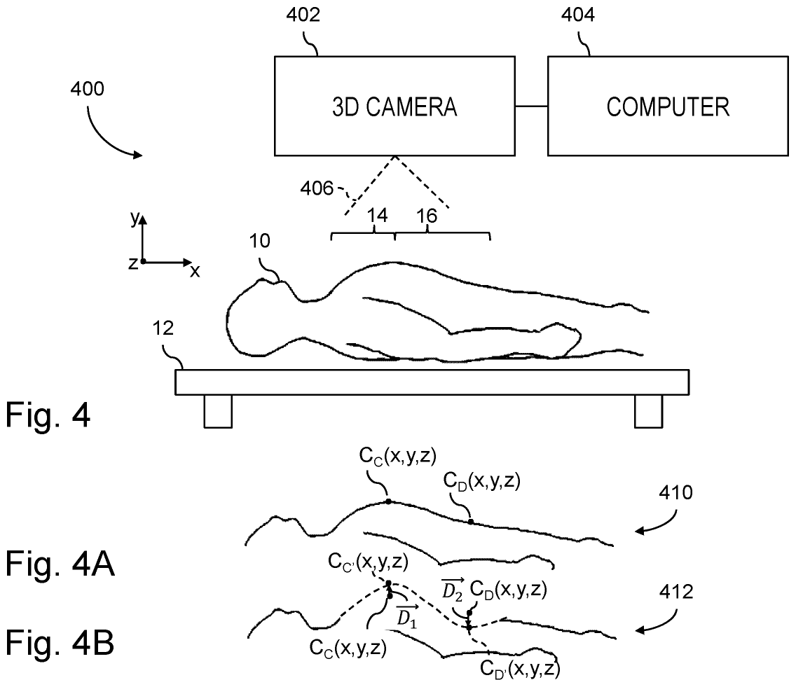

FIG. 4 shows an example of a system 400 for assessing severity of a respiratory distress of a patient 10. In this embodiment, the system 400 can be positioned proximate a hospital bed 12 on which the patient 10 lies. As depicted, the system 400 has a 3D camera 402 and a computer 404 which is communicatively coupled to the 3D camera 402.

As shown, the 3D camera 402 has a field of view 406 encompassing at least the thoraco-abdominal region of the patient 10. As such, the 3D camera 402 is used to generate one or more 3D images of the patient 10, and more particularly of the thorax and abdomen regions 14 and 16 of the patient 10. The 3D camera 402 can be a stereo camera, a structured-light 3D scanner, a movable laser range finder, an array of range finders, a time-of-flight camera, and or any other type of 3D camera. The 3D image can include, but not limited to, a cloud of points having coordinates in an arbitrary reference system (x,y,z).

Similarly to the computer 104, the computer 404 can be provided as a combination of hardware and software components. The hardware components can be implemented in the form of the computing device 200 such as shown in the example of FIG. 2.

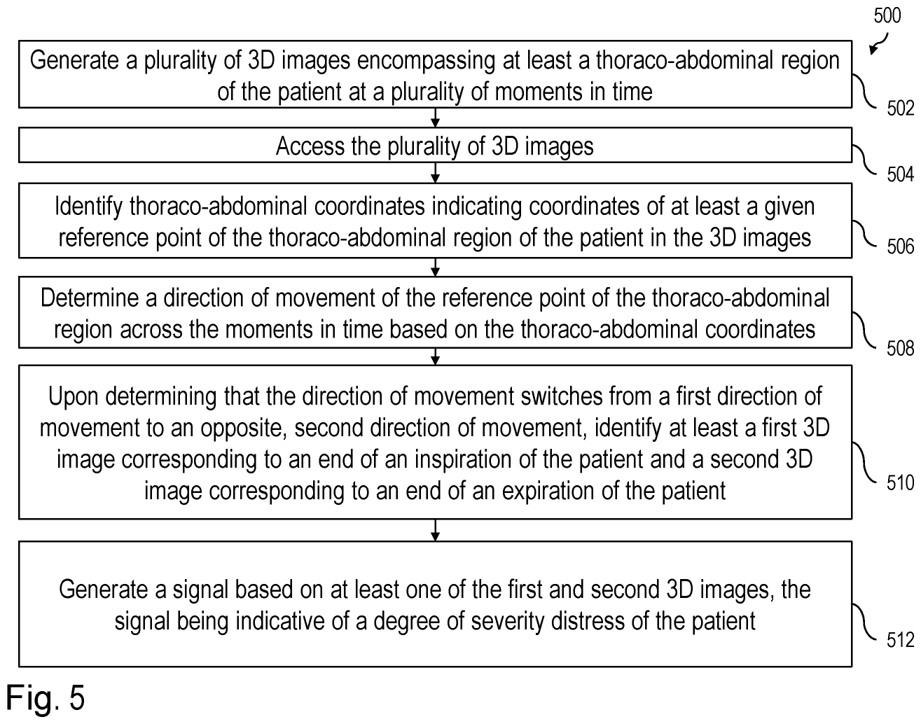

Referring now to FIG. 5, there is shown another example of a method 500 of assessing severity of a respiratory distress of the patient 10. The method 500 will be described with reference to FIGS. 4, 4A and 4B for ease of reading.

At step 502, the 3D camera 402 generates a plurality of 3D images encompassing at least a thoraco-abdominal region of the patient 10, namely the thorax region 14 and the abdomen region 16 of the patient 10 in this case. The 3D images represent the thoraco-abdominal region of the patient 10 at different moments in time as the patient breathes. The 3D images can be stored on the memory of the computer 404 or on a remote memory in some embodiments, whereas the 3D images can be communicated to a network in some other embodiments.

At step 504, the computer 404 accesses the 3D images generated at step 502. The computer 404 can access the 3D image by accessing its own memory, or a remote memory and/or by communicating with a network.

At step 506, the computer 404 identifies a plurality of thoraco-abdominal coordinates indicating coordinates of at least a point of the thoraco-abdominal region of the patient 10 in at least two of the 3D images. The two or more 3D images can be successive in some embodiments. However, the two or more 3D images may not be successive to one another, as long as the two 3D images correspond to two different moments in time. The imaged region of the patient 10 can include the thorax region 14, the abdominal region 16, or both, depending on the embodiment.

At step 508, the computer 404 determines a direction of movement of the point of the thoraco-abdominal region across the moments in time based on the identified thoraco-abdominal coordinates.

At step 510, upon determining that the direction of movement switched from a first direction of movement to a different, second direction of movement, the computer 404 identifies at least one of a first 3D image corresponding to an end of an inspiration of the patient 10 and a second 3D image corresponding to an end of an expiration of the patient 10. Indeed, it is expected that as the patient 10 ends an inspiration or an expiration, its thoraco-abdominal region will change direction. Accordingly, monitoring a switch in the direction of movement of the thoraco-abdominal region of the patient can allow to find 3D images corresponding to those moments in time.

At step 512, the computer 404 generates a signal based on at least one of the first and second 3D images. The generated signal is indicative of a degree of severity of the respiratory distress of the patient 10, if any.

In some embodiments, the point of step 506 corresponds to a first point of the thorax region 14 of the patient 10 and the thoraco-abdominal coordinates correspond to thorax coordinates and the direction of movement determined at step 508 is a first direction of movement. In these embodiments, the computer can further identify abdominal coordinates indicating coordinates of at least a second point of the abdominal region 16 of the patient 10 in the 3D images, and determine a second direction of movement of the abdominal region 16 across the moments in time based on the identified abdominal coordinates. By doing so, the computer 404 can compare the first and second directions of movement to one another. For instance, respiratory distress may be identified when the first and second directions of movement are opposite to one another. In such cases, an alert, which may be visual, auditory or tactile, may be generated. In some other embodiments, the alert may be stored on a computer memory. By performing such a comparison over time, thoraco-abdominal asynchrony of a patient 10 can be monitored over time, and detected as soon as it happens.

In some embodiments, the computer 404 may, based on the first and second 3D images, determine a retraction distance which corresponds to a distance between coordinates of a point of the thoraco-abdominal region in the first 3D image and coordinates of the same point of the thoraco-abdominal region in the second 3D image. An alert may be generated by an indicator (e.g., a visual indicator, an auditory indicator, a tactile indicator) whenever the distance exceeds a given threshold, in some embodiments. A tidal volume may also be determined by calculating a volume extending between a surface of the thoraco-abdominal region of the patient in the first 3D image and a surface of the thoraco-abdominal region of the patient 10 in the second 3D image.

As described below with Example 3, the direction of movement may include the monitoring of a curvature value evolving together with the coordinates of the point moving across the 3D images. As the curvature value increases from a 3D image to another during an inspiration or expiration, it then decreases in a successive expiration or inspiration, and so forth, which may facilitate the identification of 3D images actually corresponding to an end of an inspiration and an end of an expiration, which may be emphasized by an inflexion point in the variation of the curvature value. Additionally or alternatively, a curvature value associated with a secondary respiratory muscle may be monitored as it would provide satisfactory indication of respiratory distress in some embodiments.

For example, FIG. 4A shows a 3D image 410 of a patient 10 at a first moment in time and FIG. 4B shows a 3D image 412 of the patient 10 at a later moment in time.

As depicted, the computer 404 identifies thorax coordinates C_{C}(x,y,z) indicating coordinates of a point C_{C} of the thorax region 14 of the patient 10 in the 3D image 410 and thorax coordinates C_{C′}(x,y,z) indicating coordinates of the point C_{C} of the thorax region 14 of the patient 10 in the 3D image 412. Based on the thorax coordinates C_{C}(x,y,z) and C_{C′}(x,y,z), the computer 404 determines a first direction of movement D₁ of the point C_{C}. The computer 404 also identifies an abdomen coordinates C_{D}(x,y,z) indicating coordinates of a point C_{D} of the abdomen region 16 of the patient 10 in the 3D image 410 and thorax coordinates C_{D′}(x,y,z) indicating coordinates of the point C_{D} of the abdomen region 16 of the patient 10 in the 3D image 412. Based on the abdomen coordinates C_{D}(x,y,z) and C_{D′}(x,y,z), the computer 404 determines a second direction of movement D₂ of the point C_{D}.

As show in this example, the first and second directions of movement D₁ and D₂ are opposite to one another, thereby indicating thoraco-abdominal asynchrony. By repeating the method 500 a number of times, thoraco-abdominal synchronicity and thoraco-abdominal asynchronicity can be monitored over time.

In another aspect of the present disclosure, another method of assessing severity of a respiratory distress of a patient is presented. In this method, an emphasis is made on monitoring one or more secondary respiratory muscle of the patient. Examples of the secondary respiratory muscle includes, but are not limited to, the sternocleidomastoid muscle, the scalene muscle, and the intercostal muscle. More specifically, the method has a step of, using a 3D camera, generating at least a 3D image encompassing at least a secondary respiratory muscle of the patient. The method has further steps of accessing the 3D image; identifying secondary respiratory muscle coordinates that are indicative of coordinates of at least a point of the secondary respiratory muscle of the patient in the 3D image. In addition, the method has a further step of identifying adjacent coordinates which are indicative of coordinates of at least a point of an anatomical landmark adjacent the secondary respiratory muscle region in the 3D image. The anatomical landmark can be selected among a group consisting of: a region around a clavicle of the patient, a region below a neck of the patient and a region between ribs of the patient. Then, the method performs a step of determining a given distance and/or movement between the secondary respiratory muscle coordinates and the adjacent coordinates. Upon comparing the given distance and/or movement with a corresponding threshold, a signal is generated on the basis of the comparison, with the signal being indicative of a degree of severity of the respiratory distress of the patient.

In another aspect of the present disclosure, a method of evaluating a respiratory parameter of a patient may be performed using the systems disclosed herein. In this aspect, the 3D camera generates 3D images encompassing at least a thoraco-abdominal region of the patient at a plurality of moments in time. The 3D images may be accessed by the computer to process them in order to evaluate a respiratory parameter of the patient. Examples of such respiratory parameters can include, but not limited to, respiratory rate, tidal volume, see-saw distance, thoraco-abdominal distance, retraction distance. For instance, the evaluation step can include a step of determining a tidal volume corresponding to a volume extending between a surface of the thoraco-abdominal region in a first 3D image corresponding to an end of an inspiration of the patient and a surface of the thoraco-abdominal region in a second 3D image corresponding to an end of an expiration of the patient. The evaluation step can include a step of determining a respiratory rate of the patient. Different methods of determining the respiratory rate may be used. For example, the respiratory rate may be determined by evaluating a rate at which a point of the thoraco-abdominal region oscillates in a back and forth manner across the 3D images. In some other embodiments, the evaluation step can include a step of determining a retraction distance corresponding to a distance between a surface of the thoraco-abdominal region in a first 3D image corresponding to an end of an inspiration of the patient and a surface of the thoraco-abdominal region in a second 3D image corresponding to an end of an expiration of the patient. The respiratory parameter, which may differ from one embodiment to another, may be monitored over time. As such, alert(s) may be generated when the respiratory rate exceeds a given threshold, when the tidal volume is below a given threshold and/or the retraction distance is above a given distance. Such alerts may be displayed on a display screen or acoustically emitted near the patient's bed.

The following examples present possible embodiments of the systems and methods described above, and also expose at least some satisfactory experimental results.

### Example 1—Quantitative Assessment of Spontaneous Breathing in Children: Evaluation of a Depth Camera System

This example describes a new approach for quantitative evaluation of respiration in the pediatric intensive care unit (PICU). Video sequences of thorax movements are recorded by two depth cameras to cover the 3D surface of the torso and its lateral sides. The breathing activity implies a frame-by-frame surface deformation, which can be described by the volume variation of reconstructed surfaces between consecutive video frames. A quantitative evaluation of the breathing pattern is then performed through a subtraction technique, thereby detecting the volume variation between subsequent frames. A high-fidelity simulation was performed in a realistic environment designed for critically ill patients such as children. The simulation was then followed by a real-world evaluation, involving 2 newborn babies (1 female and 1 male) requiring the ventilator support for breathing. The breathing signal patterns resulting from this approach were compared to those measured by mechanical ventilation in terms of their waveforms, evaluating the most significant dynamic parameters: tidal volume, respiratory rate and minute ventilation. This experimental study showed a significant agreement between the proposed 3D imaging system and the gold standard method in estimating respiratory waveforms and parameters. Firstly, in this example, a 3D imaging system specifically designed for PICU based on a contactless design is proposed. Secondly, an efficient positioning mechanism for the cameras is proposed, offering a very high spatial coverage of thoraco-abdominal zone and considering the PICU constraints. Finally, an objective vision-based method is proposed to quantitatively measure respiration for spontaneous breathing patients in PICU.

Respiratory rate (RR), tidal volume (Vt) and minute ventilation (MV) can be important parameters commonly needed by doctors to assess health conditions in PICU or any other types of medical facilities, which receive children in critical condition, from newborns to 18-year-olds. These parameters are among the main indicators to determine the degree of respiratory failure. MV has a strong relationship with blood carbon dioxide levels. Patients presenting a critical life-threatening health condition, such as respiratory failure, are mechanically ventilated. For those reaching a more stable condition, most need to stay in a PICU so that medical intervention can be administered rapidly in case of sudden worsening. Their health conditions must be monitored over time to track improvements or declines. Usually, RR is measured at regular intervals of time using plethysmography, a method which can present a high rate of erroneous measures. Vt and MV can only be measured by ventilator spirometers when a child is mechanically ventilated. That said, there is currently no clinical tools to get Vt and MV measures if the child is not mechanically ventilated.

There remains a need for reporting quantitative measures of minute ventilation using a contactless method. Secondly, there are challenges in accommodating the clinical environment, specifically the PICU, because of their paucity or absence of quantitative measures as well as the complexity of their system's setup. It is believed that this is the first work that reports quantitative measures of respiratory rate, tidal volume and minute ventilation together in a PICU. Most importantly, these measurements can be obtained when the patient is not mechanically ventilated.

In this example, two Time-of-Flight (ToF) cameras have been used to perform a surface reconstruction of the upper part of the torso and its lateral sides. This has been successfully achieved through an efficient positioning mechanism for the cameras, offering a very high spatial coverage of thoraco-abdominal zone for a good surface reconstruction. The volume variation change between consecutive reconstructions is then calculated. From the volume variation, we extract quantitative measures of respiratory rate, tidal volume and minute ventilation together in a pediatric intensive care room. Most importantly, these measurements can be obtained when the patient is not mechanically ventilated. Furthermore, the system components accommodate the PICU room and can be easily and quickly detached from the bed allowing the urgent transport of the patient in emergency cases.

The acquisition detailed setup, the cameras registration, the surfaces reconstruction and detailed algorithm were described and discussed in the following paragraphs. An RGB-D sensor is able to capture three simultaneous streams: Color (RGB), Depth (D) and Infrared Radiation (IR).

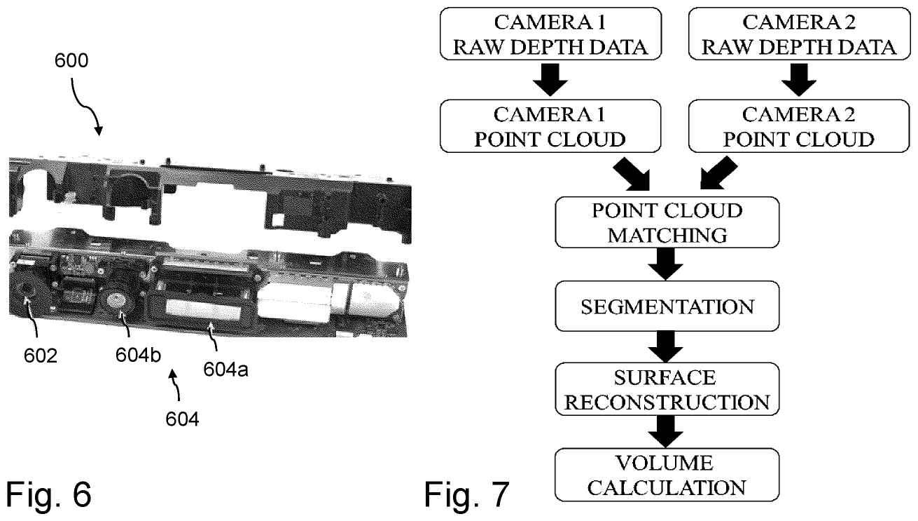

FIG. 6 illustrates an example of animaging system 600, in accordance with an embodiment. The imaging system 600 has a first camera system including a RGB camera 602, and a depth sensor 604 incorporating an infrared emitter 604a and an infrared camera 604b) to acquire color, infrared and depth images of the scene. The color data arise from the RGB camera 602, while infrared data and depth maps come from the depth sensor 604 and have the same resolution. The imaging system 600 can have an additional, second camera system similar to the first camera system shown in FIG. 6 The color data have a very high-resolution of 1920×1080 pixels (px) in this example. The depth maps of inferior resolutions (512×424 px) are 2D images, where depth information is stored for each pixel. To estimate depth, the imaging system 600 uses the time-of-flight technique by measuring the round-trip time needed by a light pulse to travel from the sensor illuminator to the target object and back again. The illuminator is a near-infrared laser diode emitting a modulated infrared signal to the object. The reflected light is collected by the sensor detector. A timing generator is used to synchronize the actions of the emitter and the sensor detector. The depth of each pixel is then calculated by Equation (1):

$$d = \frac{Δφ}{4 π f} \times c , \qquad ( 1 )$$

where d is the distance to be measured (pixel depth), Δφ is the phase shift between the emitted light and the reflected light, c is the speed of light (3×108 m/s) and f is the modulation frequency.

In this example, the first and second camera systems are used to capture the scene from two viewpoints simultaneously and automatically merge them. Once the views are aligned, a region of interest (ROI) is segmented. The ROI includes the body region surface involved in breathing from two angles of view, allowing a high coverage. The ROI surface is then reconstructed in order to calculate the volume at Frame t. FIG. 7 illustrates the process of the respiratory parameters calculation, starting from raw depth data acquisition and leading to the volume calculation at a given frame. The proposed system then calculates a volume time-curve from the calculated volumes in subsequent frames. Vt and RR are finally estimated from the volume-time curve.

Point clouds are a set of points in the 3D space used to create a representation of a scanned physical object. Points in a point cloud are always situated on the external surfaces of the object. They are very useful for 3D modeling and remain the starting point in any 3D data processing application. A point cloud derives from raw data. Indeed, it is straightforwardly generated from depth data using the camera software development kit (SDK). In this approach, point clouds need to be available simultaneously from two different view angles to provide a high spatial coverage of the patient's torso. Accordingly, point clouds alignment in a same coordinate system is performed as a first step in the proposed method. This can be performed by aligning the camera systems to a common marker. The proposed method assumes that the first and second cameras have a common view zone where the common marker can be easily detected by both camera systems. Each point cloud, covering a section of patient's torso, is thus aligned in the common coordinate system using the transformation matrix in Equation (2).

$$M = ( \begin{matrix}cos {θ}_{y} cos {θ}_{z} & sin {θ}_{x} sin {θ}_{y} cos {θ}_{z} + cos {θ}_{x} sin {θ}_{z} & - cos {θ}_{x} sin {θ}_{y} cos {θ}_{z} + sin {θ}_{x} sin {θ}_{z} & {t}_{x} \\ - cos {θ}_{y} sin {θ}_{z} & - sin {θ}_{x} sin {θ}_{y} sin {θ}_{z} + cos {θ}_{x} cos {θ}_{z} & cos {θ}_{x} sin {θ}_{y} sin {θ}_{z} + sin {θ}_{x} cos {θ}_{z} & {t}_{y} \\ sin {θ}_{y} & - sin {θ}_{x} cos {θ}_{y} & cos {θ}_{x} cos {θ}_{y} & {t}_{z} \\ 0 & 0 & 0 & 1\end{matrix} ) \qquad ( 2 )$$

In fact, each of the first and second camera systems infers its relative position from the detected marker, which represents the world coordinate system. This presumes the estimation of two matrices _{C1}^{W}M and _{C2}^{W}M from the cameras coordinate systems to the world coordinate system. In Equation (2), the transformation matrix has six variables (θₓ, θ_{y}, θ_{z}, tₓ,t_{y},t_{z}). It can be expressed as combinations of three parameters coming from 3D translation (tₓ, t_{y}, t_{z}) and three other parameters coming from 3D rotation (θₓ, θ_{y}, θ_{Z}). By calculating the rotation R and the translation t, find the transformation matrix can be found. To find the optimal transformation, the Procrustes analysis was used as it is recognized for its effectiveness to resolve these types of problems. Procrustes analysis is the process of superimposing one collection of marker configurations on another by translating, scaling, and rotating them, so that the distances between corresponding points in each configuration are minimized. The Procrustes distance is based on a least-square fit method and requires two aligned shapes with one-to-one point correspondence.

The process of superimposing a marker on another is divided into five main steps: marker detection, finding centroids, marker scaling, finding rotation and translation, and finally Procrustes distance computing. The first step uses only color data to detect the marker with a simple thresholding applied on the input images. The number of vertices of the detected area is compared to the number of vertices of the known shape to eliminate false results. If many images are detected, a subpixel precision processing technique is applied to refine the marker vertex locations. The second step uses the geometric model of the marker and computes its center of mass, so that the target marker can be placed over the reference configuration. In the third step, differences in size between configurations are removed by rescaling each configuration. Then, the differences in orientation is achieved by rotating one configuration (the target) around its centroid until it shows minimal offset in location of its landmarks relative to the other configuration (the reference). To transform a detected shape by the camera X₁=(x₁₁, x₂₁, . . . xₙ₁)^{T} to an already known shape X₂=(x₁₂,x₂₂, . . . xₙ₂)^{T}, Equation (3) was used:

X₂=R×X₁+t (3)

where R is the rotation and t is the translation.

To compute the Procrustes distance between the target and reference structures, equation (4) was applied:

P_{d}²=Σⱼ₌₁ⁿ[(xⱼ₁−xⱼ₂)²+(yⱼ₁−yⱼ₂)²] (4)

These steps are repeated in order to minimize P_{d}² and subsequently compute the optimal alignment.

The extraction is performed using Cloud Compare and Point Cloud Library. By including classical computer vision functions and algorithms, Cloud Compare allows 3D data processing and visualization. The contributor community is growing and expanding its applications in many research and industry fields. As such, Cloud Compare is continuously updated and becoming a standard tool in 3D data processing. Cloud Compare uses the Point Cloud Library as a third-party library to provide a set of additional computer vision algorithms, such as 3D data filtering, projections, feature estimation, etc. Point Cloud Library is a C++ library containing various algorithms to process all forms of point cloud data. This includes color data, depth data, point clouds, mesh data, noisy data and even reconstructed models. Point Cloud Library also includes numerous filters for data cleaning. These filters can process the data based on the position of the points in addition to other parameters. For example, some Point Cloud Library filters can be used to drop any points with an intensity value below a certain threshold. In this example, the 3D vision libraries are used for extracting the region of interest, as well as for cleaning the point cloud.

Once point cloud matching is performed, a rectangular cuboidal region of interest (ROI) is extracted including the thoraco-abdominal region using Cloud Compare. The clouds are selected at once and then aligned together. The proposed imaging system is positioned in a manner to ensure the inclusion of the thoracic-abdominal area in the extracted region. It should be noted that precise segmentation of the thoracic-abdominal region is not performed by finding its boundaries. Instead, a coarse segmentation is performed by extracting a rectangular cuboid including the thoracic-abdominal region. Since the proposed method for volume calculation is based on a subtraction technique, a precise segmentation of the ROI is not needed and only the moving volume due the chest contraction and expansion between subsequent frames is retained. The rest of volume is removed by the subtraction operation. Moreover, the coarse extraction technique allows a significant decrease of the computation time. The extracted 3D point cloud may contain noise that appears as clusters of neighboring points. This noise is removed using the Statistical outlier removal filter of the Point Cloud Library. This filter allows removing points that do not statistically fit with the rest of the data. The principle is to calculate the mean distance from each point to all its neighbors. The distribution is assumed to be Gaussian with a mean and standard deviation. Then, a threshold value is computed based on the mean and standard deviation of all distances. The filter finally keeps points whose mean distance is below the threshold value.

Because of the presence of holes and surface discontinuity, the point cloud information is not sufficient to calculate the volume. An intermediary mesh with closed gaps then needs to be generated. Using meshes simplifies surface reconstruction significantly. Thus, the surface reconstruction scheme follows three essential phases. Once the surface is scanned and the point cloud is calculated, a minimum spanning tree propagation technique is applied in order to compute and orient normals, equivalently referred to as vectors perpendicular to their curve. In this case, this technique allows to close the reconstructed surface. Its main principle consists in constructing a graph over the point cloud for all the vertexes through the k-nearest neighbors of each point. Then, the orientation of the vertex with the highest z value is calculated. Afterward, the correction of the direction of the entire vertex is conducted across the graph. Finally, the surface is reconstructed using Poisson surface reconstruction, which takes as input a group of points with oriented perpendicular vectors and calculates a closed volume. By acting on a set of 3D points with perpendicular vectors, the method solves for an approximate indicator function of the inferred solid, whose gradient best matches the input perpendicular vectors. The indicator function is zero everywhere except close to the surface. Note that all surfaces are closed by considering a reference plane at a well-defined distance from the subject's back and the lateral chest wall.

The volume of the reconstructed surface is calculated using Cloud Compare. The proposed method relies on the octree 3D structure representation. Based on a hierarchical tree structure, an octree partitions the 3-D space. Starting from a root node in the form of single large cube, the octree is recursively subdivided into eight equal sized sub-cubes. This subdivision process continues until a predefined maximal depth is reached or if the regions are empty. The final volume is computed for each frame by multiplying the number of octrees by the unit size.

As a result, a 1D signal is computed where the frequency is the respiratory rate. On the other hand, the change in the signal amplitude is the key to estimate the tidal volume Vt. Note that the position of the reference plane may not be important in Vt estimation, as even if the real volume of the thoracic-abdominal area is not accurate, this does not affect the accuracy of volume difference between frames with the subtraction method. The ROI volume is calculated at each frame to estimate a surrogate of patient's real volume-time curve. After detecting relevant peaks and minima of the curve, the tidal volume is deduced by subtracting volume values corresponding to consecutive extrema points. On the other hand, the respiratory rate is calculated from the volume-time curve by simply counting the number of peaks in a minute. In fact, each cycle has only one peak corresponding to the end of an inspiration.

To improve the accuracy of the proposed method, the average duration of a respiratory cycle (D) is computed using Equation (5):

$$D = \frac{{Σ}_{i = 1}^{{N}_{p} - 1} {d}_{i}}{{N}_{p} - 1} , \qquad ( 5 )$$

where Nₚ is the number of peaks of the volume-time curve in a minute and dᵢ is the temporal distance between peaks i and i+1.

The respiratory rate RR is then deduced using Equation (6):

$$R R ( respirations / minute ) = \frac{60 seconds}{D} \qquad ( 6 )$$

The tidal volume is the volume of air inhaled or exhaled from a person's lungs in a cycle. For more accuracy, the final tidal volume in a cycle is calculated as the average value of inspiratory and expiratory volumes. The tidal volume per minute is thus the average of all tidal volumes during a minute as shown in Equation (7):

$$V t = \frac{{Σ}_{i = 1}^{{N}_{p}} t {v}_{i}}{{N}_{p}} , \qquad ( 7 )$$

where tvᵢ is the tidal volume of the cycle i.

To simulate the breathing activity, a baby mannequin designed according to neonatal anatomical and physiological characteristics was used, and with an artificial test lung for infants (MAQUET Medical Systems, 1 Liter Test Lung 190). The lung is branched to a mechanical ventilator (Servo i, Maquet Inc, Sweden). The ventilator is a bedside machine used to push a volume of air into the lungs. The pushed volume is usually adjusted by caregivers according to the baby's weight and condition.

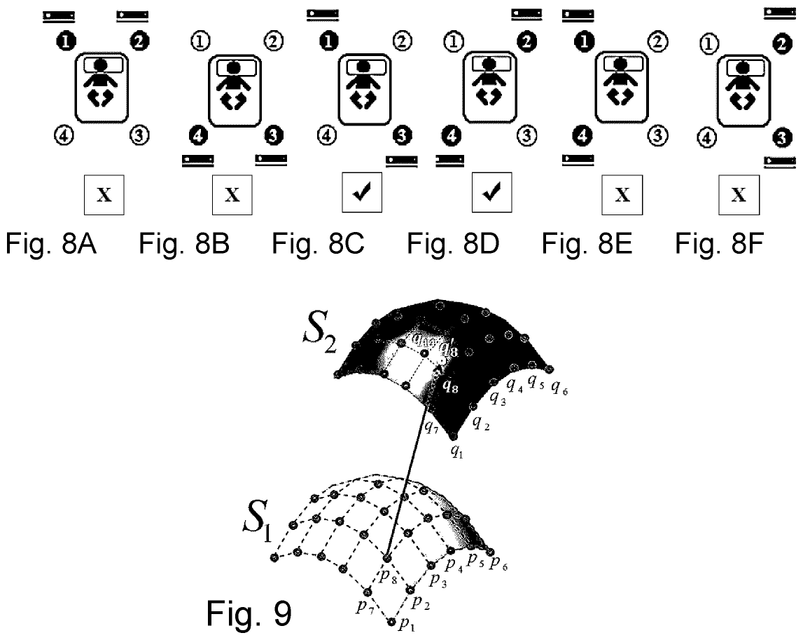

The first and second camera systems can be disposed according the different schemes shown in FIGS. 8A-8F. Considering the limited space in a PICU, the cameras can be placed on two of the four legs of the bed. Since the knowledge of lateral surface motion is important for a complete torso reconstruction, the mannequin's lateral sides should be covered by the field of view of the two cameras together. In FIGS. 8A-8F, all possible combinations are illustrated. Only the four first configurations are advantageous (FIGS. 8A, 8B, 8C and 8D), as the other configurations do not allow coverage to both lateral sides. These first four positions were tested experimentally and only positions shown in FIGS. 8C and 8D were retained. In fact, the depth sensor is placed on the left side of the camera as illustrated in FIG. 6. On this basis, depth views are not symmetrical. In configuration shown in FIG. 8A, the camera placed at the right of the patient (camera 1) allows to have a good point cloud of the right lateral side of the torso whereas the left camera (camera 2) does not cover the left side of the torso due to the position of the infrared sensor. In configurations shown in FIGS. 8C and 8D, both cameras allow good point clouds of both lateral sides. The sensors are finally positioned in the top right and the bottom left of the bed (configuration depicted in FIG. 8C), both in the direction of 45° and at a distance of 1 m to the crib mattress. This positioning offers a high spatial coverage since the top and lateral sides of the baby are covered.

For system calibration, the 2D marker is placed on the bed in such a way to be in a common field of view of the two cameras. The cameras infer their relative positions from the detected marker. The marker was then removed and the baby mannequin was placed in the bed.

In order to evaluate the performance of the proposed method, the ventilator is used as gold standard. In PICE and for health professional decision-makers, the ventilator is considered as the most reliable method to provide accurate and precise quantitative measures for RR and Vt. Thus, ventilator measures are recorded in parallel to the experiments and are considered as ground-truth data.

In this example, spontaneous breathing of a patient was simulated with different volumes. Note that the mannequin lung supports volumes from 10 mL to 1 L. Therefore, the same mannequin was used to test different volumes for all ages. Two primary modes were used to push the air into the artificial lungs: the neonatal and the adult mode. The air volumes for neonatal mode are respectively: 10 ml, 20 ml, 30 ml, 40 ml, 50 ml and 100 ml. For adult mode, the volumes are respectively: 150 ml, 200 ml, 250 ml, 300 ml, 350 ml, 400 ml, 450 ml and 500 ml. Vt and RR are computed with the proposed method. The results are then compared with the ventilator reference values. To verify the applicability of the proposed method on a real patient, a second test was conducted by measuring the breathing pattern of a mechanically ventilated infant. This test involved a 4 months and 20 days old female, weighed 6.6 kg. The patient was sleeping and requiring the ventilator support for breathing. The test was performed in a PICU room of Sainte-Justine Hospital, one of the largest pediatric health centers in Canada. This experiment was conducted with approval from the Research Ethics Board (REB) of the hospital. Kinect camera systems were placed to accommodate the patient and the already existing medical equipment. In case of emergency, the camera systems can be easily and quickly detached from the bed allowing the urgent transport of the patient. This configuration was checked and validated by the equipment inspection team of the Hospital.

Note that the breathing activity can be controlled totally or partially by the mechanical ventilator. For example, the ventilator performed the entire breathing activity in the first test with the mannequin. In the second test (with a real patient), the ventilator is doing the preponderance of the breathing work, while the patient is partially contributing in the respiration. The ventilator settings are set to Vt=40 mL and RR=20 respirations/minute. The final Vt and RR values displayed by the ventilator are not only controlled by the ventilator, but also by the patient's breathing effort.

The common Euclidean distance [symbol] ₂ is adopted to calculate distance between clouds. S₁ and S₂ were considered the external surfaces respectively in the initial and final state (before and after being inflated with air), as indicated in FIG. 9. Point clouds of the surface S₂ are regarded as “target” points q=(qₓ,q_{y},q_{z}), whereas the point clouds of the surface S₁ are considered as points p=(pₓ,p_{y},p_{z}) in the “initial position”. The distance between p and q is calculated using [symbol] ₂ in the space [symbol] ³. The aim is to find corresponding 3D points before and after surface displacement from S₁ to S₂. Consider that M source points cloud pᵢ are provided on the surface S₁. Points pᵢ, i∈{1 . . . M} from S₁ are projected on S₂ using the normal vector at each source point. The projected points are noted qᵢ′, i∈{1 . . . M}. To find a corresponding destination point in S₂, the nearest neighbor is selected in qᵢ, i∈{1 . . . M}. Then, the displacement distance is computed for each pair in the cloud using Equation (8), where p represents the “initial” point in S₁ surface and q is the “target” point in S₂ surface.

∥p−q∥=\sqrt{(pₓ−qₓ)²(p_{y}−q_{y})²(p_{z}−q_{z})²} (8)

The maximum displacement is selected for each cloud. For each experiment, these steps are repeated over each pair of Nₚ clouds. To compute the maximum displacement Δd in each experiment, Equation (9) was used:

Δd=max_{j∈[1,N}_{ₚ}_{]}(max_{i∈[1,M]}(∥pᵢ^{j}−qᵢ^{j}∥)) (9)

where the maximum displacement is first calculated over one pair of point cloud and then calculated over the Nₚ clouds of each experiment.

In FIG. 9, the source point p₈ on the surface S₁ (before displacement) is projected on the surface S₂ (after displacement) using the normal vector in p₈. As can be seen, the nearest neighbors of the projected point q′₈ are q₈ and q₁₄. Since q₈ is closest to q′₈ than q₁₄ as ∥q₈′−q₁₄∥>∥q₈′−q₈∥, it is selected as the corresponding point of p₈. Finally, the depth displacement distance is computed for the pair (p₈, q₈) by calculating ∥p₈−q₈∥.

The maximum displacement Δd is computed for different combinations of ventilator Vt settings.

In the above, it was demonstrated that it was possible to track torso volume changes. These results have been validated by evaluating the root mean square deviation (RMSD), the relative error (RE) and the relative standard deviation (RSD) metrics applied on RR and Vt measures. In this example, there is presented an extensive validation of the imaging system using several improvements of the respiratory assessment algorithm, the measurement of a new parameter which is the minute ventilation (MV), an extensive experimental work and real patient's data.

More specifically, an in-depth study of volume—frame curve to extract key points for quantitative breathing assessment has been performed. There is also described a method for calculating a minute ventilation parameter in spontaneous breathing, which is a good indicator for carbon dioxide level in the blood. Experiments investigating the performance of the 3D video system have also been conducted. The experiments were performed for simulated controlled scenarios using a high fidelity phantom simulator with different pediatric volumes and for real uncontrolled scenarios conducted on two PICU children requiring the ventilator support for breathing. An evaluation of the proposed method using a statistical analysis and method-comparison study where the agreement between the proposed method and mechanical ventilation has been studied, a reference method currently used in intensive care environments. Results are presented with regression analyses, as well as with the Bland-Altman (BA) plots, two evaluation methods that are commonly used in the medical field.

Compared to its previous model (Kinect v1), the camera presents a better resolution for the raw depth data stream (512×424 pixels for Kinect v2 versus 320×240 pixels for Kinect v1) and a higher field of view (70°×60° for Kinect v2 versus 57°×43° pixels for Kinect v1). Moreover, it was suggested that Kinect v2 depth resolution are 2 mm under 3 meters' distance. Accordingly, valid signals can be obtained for detecting surface movements with small amplitudes in the range of few millimeters. The imaging system has been considering the use of two Kinect v2 camera systems for providing motion information with high spatial coverage of the respiration zone. For each Kinect camera system, the acquired depth information is processed and converted to a point cloud. A point cloud is a data structure in the form of an array of points, with each cell containing the x, y and z coordinates for a specific point. Derived from depth data, a point cloud represents the external surface of the scanned object and is the starting point in many 3D data processing applications. Using the Kinect for windows software development kit, point clouds are directly generated from depth data.

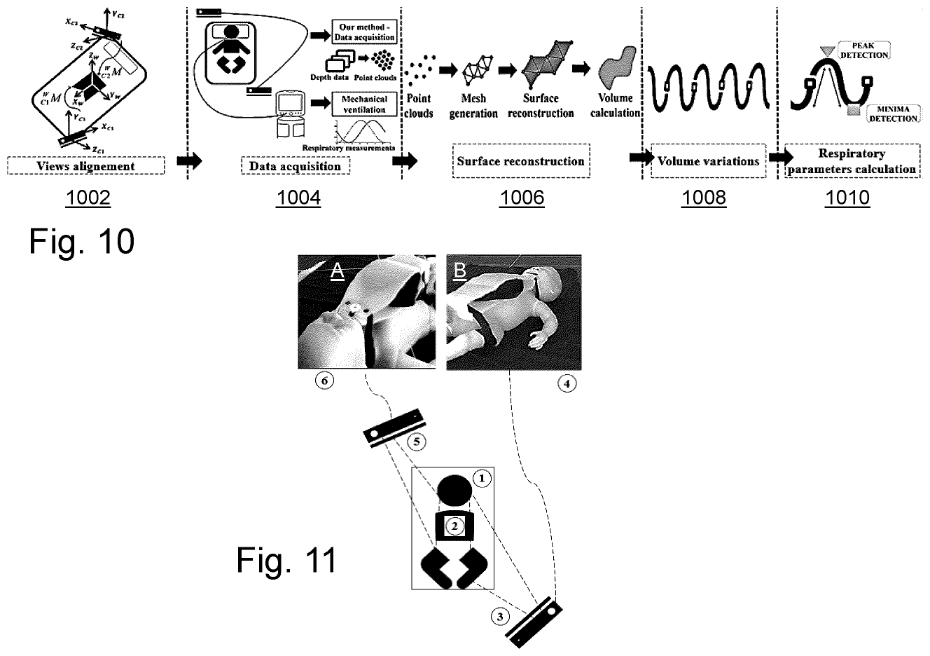

FIG. 10 shows an overview of the proposed computer vision system at different steps of the method disclosed herein. As best shown at step 1002, the viewpoints of the cameras are first aligned in a common coordinate system. Then, at step 1004, two sets of data are simultaneously collected by simulating the breathing activity. The first set is the depth data acquired by the proposed system from two complementary view angles, while the second set corresponds to the mechanical ventilator parameters. This second set can be used for the validation of the proposed method. The first set of depth data is transformed into a point cloud using the framework functions, after a region of interest has been identified and extracted. At step 1006, surface are reconstructed from the clouds of points generated by the cameras. Volume can be calculated from the reconstructed surface, for instance. Upon monitoring the calculated volume, at step 1008, respiratory parameters can then be calculated at step 1010.

As discussed above, the proposed system has two opposite Kinect camera systems which can be mounted on two adjustable length metal stands, which are PICU bed accessories, originally used as serum hanger (IV Pole). The two metal stands are placed in the top right and the bottom left of the patient bed in one exemplary embodiment. It was found convenient to position the two camera systems in a stabilized manner at a height of 100 cm above the crib mattress and tilted down at 45 degrees from the horizontal position. The second version of the Microsoft depth sensor model (Kinect v2) has been used in this example for its remarkable technical properties such as spatio-temporal resolution.

In this method, point clouds need to be available from two camera systems presenting complementary view angles. The final view can include the information for the top of the torso movement as well as for its lateral sides, as shown in FIG. 11. To position the camera to offer this full view, the position and orientation of the camera are determined in a world reference frame given a set of points and their corresponding 2D projections in the image. The camera position and orientation consists of a transformation matrix with 6 degrees-of-freedom (DOF) which are made up of the 3D translation and the rotation (roll, pitch, and yaw) of the camera with respect to the world. Each camera can infer its relative position in the world coordinate system using the transformation matrix.

To find the optimal transformation, Procrustes analysis (PA) was used, as it is known as an efficient method in shapes comparison by removing rigid transformations between them. The transform parameters between two shapes (a detected shape by the camera and a reference shape) was calculated by matching them to be as close as possible. For this purpose, the detected shape was translated, scaled and then rotated towards the reference shape. A five-sided polygon may be used to find the optimal transformation between the camera coordinate frame and the world coordinate frame. The marker location is found using thresholding in a first step. For more precision, the number of vertices of the detected polygon is compared with the number of vertices of the reference shape. Once the marker vertices are matched between the reference and detected shapes, the corresponding metric locations are found using the provided Kinect software development kit. In the second step, the center of mass of both detected and reference shapes was computed to align them at a common centroid. In the third step, detected shapes were rescaled to have an equal size with the reference shapes. Then, the difference in orientation between two shapes was reduced by rotating the polygon around its centroid until a minimal distance between the shapes is realized. To illustrate these steps, equation (10) was used:

X₂=R*X₁+t, (10)

where X₁ denotes the detected shape and X₂ denotes the reference shape, R denotes the applied rotation and t denotes the applied translation.

To compute the Procrustes distance between the target and the reference structures, equation (11) was applied, where the sum of squared distances was minimized with one-to-one point correspondence.

P_{d}²=Σⱼ₌₁ⁿ[(xⱼ₁−xⱼ₂)²+(yⱼ₁−Yⱼ₂)²]. (11)

The alignment procedure can include a 2D marker which is aligned in two different views, each one of them covering an area of the respiratory zone. The final point cloud includes the complete information of the torso and its lateral sides.

After point cloud alignment is accomplished, the surface reconstruction can be performed. First, each cloud is properly cleaned of any noise and outliers using the Statistical Outlier Removal filter (SOR) of the Point Cloud Library (PCL). To simplify the computation, a ROI was extracted, the ROI including the thoracic-abdominal area, using the software Cloud Compare (CC). The clouds are selected and then segmented together all at once. The segmented thoracic-abdominal area does not have to be precise, as the proposed method is based on a subtraction technique. Following the segmentation of the ROI, the volume variations due to the surface motion can only be those resulting from the chest contraction and expansion between successive frames.

To compute the volume, a closed surface is required. However, creating good surfaces from scanned objects is a complex task for which traditional modeling techniques have proven to be challenging. A closed surface was created by means of five main steps: (1) generating mesh from point clouds, (2) removing artefacts and fixing holes, (3) closing meshes by using a reference plane, (4) computing and orienting normals, and (5) applying the Poisson reconstruction method. First, a mesh with closed gaps needs to be generated from point clouds. Using meshes considerably simplifies surface reconstruction. Having holes or gaps in the mesh is one of the most common errors that prevent an accurate surface reconstruction and give an invalid volume. Artifacts were removed and holes were filled using a known reconstruction algorithm. The mesh is then closed using a reference plane placed at the patient's back. The minimum spanning tree technique was used to compute and orient perpendicular vectors. This method was found to be convenient when the surface is open. The idea is to construct a graph over the mesh using the k-nearest neighbors' algorithm and to estimate the orientation of the top of the graph. Then, the graph was inspected and the orientation of all the vertexes was corrected. Finally, the Poisson reconstruction method was applied, known for its efficiency in surface reconstruction, to compute a closed volume. Acting on a closed mesh with oriented perpendicular vectors, a 3D indicator function x of the inferred solid was computed whose gradient best matches the input perpendicular vectors. This function is equal to zero everywhere except close to the surface. The reconstructed surface was obtained by extracting a suitable isosurface.

The volume is calculated by subdividing the reconstructed surface using an octree representation, a hierarchical tree data structure that offers a high performance. Beginning from a root element, the octree is recursively subdivided into eight equal sized sub-cubes. The root octree element is a large 3D cube covering the reconstructed surface. This subdivision continues until a maximal octree depth is achieved or if the octrees are empty. The final volume is then calculated in each reconstruction by multiplying the octrees number by an octree unit size.

After the volume is obtained, the volume variations are represented in the form of a 2D signal whose frequency is the respiratory rate and whose maximum-to-minimum amplitude difference is the tidal volume. In fact, the respiratory rate can be calculated by simply counting the number of peaks in a minute. Each peak corresponds to the end of an inspiration.

However, to improve the accuracy of the proposed method, equation (12) is used:

$$R R ( \frac{respirations}{minute} ) = \frac{N * 60}{Δ T} , \qquad ( 12 )$$

where RR, expressed as the number of respirations per minute, denotes the respiratory rate, N denotes the number of peaks of the volume-time curve during the observation time ΔT (in seconds).

To compute the average tidal volume in a minute, equation (13) is used:

$${V}_{t} = \frac{{Σ}_{i = 1}^{N} t {v}_{i}}{N} * \frac{60}{Δ T} , \qquad ( 13 )$$

where tvᵢ is the tidal volume of the cycle i.

The minute ventilation (or pulmonary ventilation) was also computed, which is the volume of air inspired or expired during one minute, as given by equation (14):

$$M V = R R * {V}_{t} = {\sum }_{i = 1}^{N} {tv}_{i} ⋆ {( \frac{60}{Δ T} )}^{2} \qquad ( 14 )$$

The inspiratory time is the amount of time taken to deliver the tidal volume of air to the lung. To compute the average inspiratory time, equation (15) was used:

$$T I = \frac{{Σ}_{i = 1}^{N} t {i}_{i}}{N} * \frac{60}{Δ T} , \qquad ( 15 )$$

where tiᵢ denotes the inspiratory time of the cycle i.

In the experiment, two sets of data are collected simultaneously by simulating the breathing activity in an intensive care room at Sainte-Justine Hospital in Montreal. The first set of data corresponds to the quantitative measures obtained using the proposed method while the second set corresponds to those of the gold-standard method, the “Mechanical ventilation” method. The equipment was designed and adjusted to minimize the space it occupies in the room. This equipment includes the acquisition devices (two cameras) and the objects utilized to simulate spontaneous breathing. The two cameras are installed on two sides of the patient's bed, at its top and bottom, in opposite positions and pointing towards the chest. This allows breathing information to be collected for the torso surface and its lateral sides. The objects used to simulate spontaneous breathing consist in an artificial test lung for children (MAQUET Medical Systems, 1 Liter Test Lung 190), placed over torso region of a phantom designed according to neonatal anatomical and physiological characteristics and connected to a mechanical respirator (Servo i, Maquet Inc, Sweden). The respirator is a bedside machine insufflating a volume of air into artificial lungs. The insufflated volume is fixed by doctors during experiments according to the patient's ages and weights.

Two primary modes were used to simulate spontaneous breathing: neonatal and adult. A clinician participated in the acquisition and selected the different volumes for both modes. The ventilator is set to the volume controlled ventilation (VCV) mode, in which breaths are delivered based on set variables. Three variables were adjusted for each experiment on the ventilator screen: the respiratory rate, the tidal volume and the inspiratory time. These parameters vary from patient to patient according to their age and weight.

To further assess the precision of the proposed method, an analysis was carried out based on repetitive testing. Each experiment is repeated 5 times under unchanged conditions. The clustered observations were analyzed based on the four parameters (tidal volume, respiratory rate, minute ventilation and inspiratory time) using the Bland-Altman method.

In this example, the agreement between the proposed method and mechanical invasive ventilation (gold standard) was studied in terms of respiratory rate and tidal volume measurement. Mechanical ventilatory support is based on ventilator spirometry and is routinely used as life-sustaining treatment for critically ill patients in intensive care. The main principle of a mechanical ventilator is to deliver into the lung either a defined volume (which creates a positive intra-thoracic pressure) or a defined pressure (which generates a variable volume depending on the respiratory system compliance and resistance). In this example, the volume controlled ventilation mode was chosen with a known volume. Indeed, the volume is pre-defined for each experiment, so that a direct comparison can be made between measures.

Other studies have modelled the respiratory system as a linear model using equation (16):

$${P}_{a w} + {P}_{m} = {R}_{r s} V + \frac{\dot{V}}{{c}_{rs}} + P E E P , \qquad ( 16 )$$

where P_{aw} is the airway pressure of the respiratory system, Rᵣₛ is the airway resistance, Pₘ is the impact of respiratory muscle, Cᵣₛ is the degree of lung expansion per unit pressure change called lung compliance, and PEEP is the positive end expiratory pressure, which is the pressure in lungs above the atmospheric pressure outside the human body.

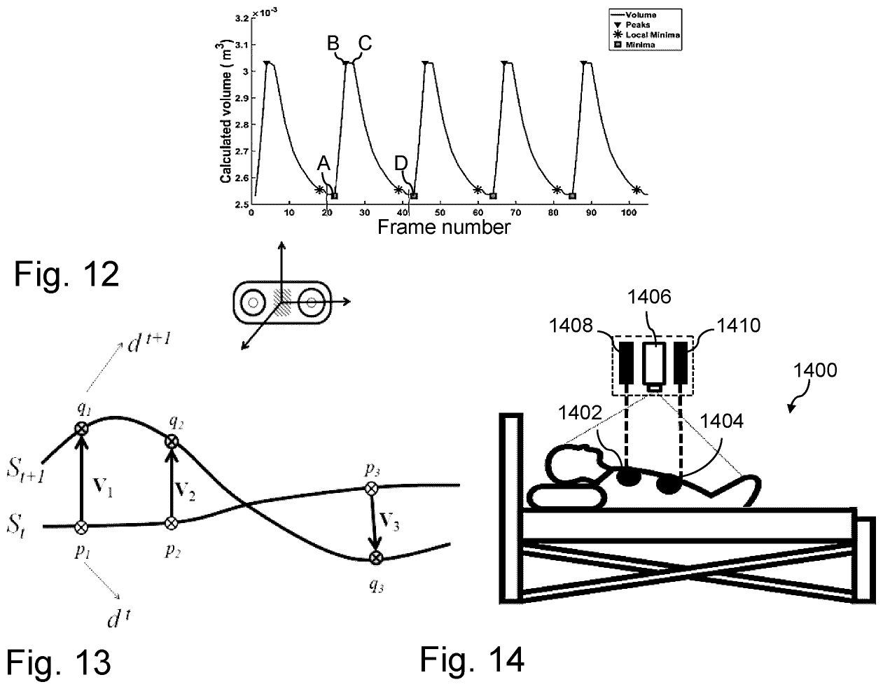

The proposed method estimates quantitative measures from the volume variation of the 3D reconstructed surface. FIG. 12 shows the volume variation calculated using the proposed method for the first five cycles. Data were collected by the proposed method during one minute for each experiment. The ventilator is set to volume controlled ventilation mode with fixed ventilation parameters (tidal volume: 500 ml, respiratory rate: 20 respirations/minute and inspiratory time: 0.9 seconds). From FIG. 12, it can clearly be seen that volume variation is a periodic signal as it completes a pattern within a measurable time frame. This pattern corresponds to one cycle-breath. Cycle 2 is represented on a larger scale at the top of FIG. 12 (restrained values of x-axis between frames number 20 and number 42). The tidal volume is the average value of the inspiratory volume (A-B) and the expiratory volume (B-D), and the inspiratory time is represented by the number of frames between the start of inspiration (reference point A) and the end of inspiration (reference point C).

The reference Vt, RR and MV were obtained from ventilator measures. Their values were respectively estimated in milliliters (mL), breaths per minute (breaths/minute) and liters per minute (L/minute), using five one-minute experiments repeated five times. The first set of experiments was performed using a high-fidelity mannequin with known breathing children patterns and not with real patients. The tested patterns include different pediatric volumes from 10 mL to 500 mL. The phantom experiments were followed by two real patients' experiments to confirm the suitability and adaptability of the proposed system to real patients. The first child is a 4 months old female having a weight of 6.6 kg weight. The second child is a 1 year old male having a weight of 13.4 kg. Mechanical ventilation provides full or partial support during the breathing activity. Indeed, the respiration is completely controlled by the ventilator in phantom experiments, and partially controlled by the ventilator in real patients' experiments. The second patient was making more breathing efforts than the first patient, and was, thus, more assisted in his breathing activity by the ventilator.

To measure the performance of the introduced algorithms for Vt, RR and MV estimation, root mean square deviation (RMSD) was used. Regression analyses, as well as the Bland-Altman (BA) plots, were used to assess the associations and agreements between the proposed system and ventilator measures. All the tests were conducted at a 95% confidence level. Values of the no-correlation coefficient p<0.05 were taken to be significant.

The resulting RMSD between measured and reference values shows an error of 8.94 mL, 1.36 breaths/minute and 0.2 Liters/minute for respectively Vt, RR and MV (see Table 3). These small RMSD values indicate that the quantitative measures of the proposed method are very close to those given by the gold standard method. Hence, it was found that the proposed method presents a satisfactory accuracy in estimating Vt, RR and MV.

### Example 2—Visualizing and Quantifying Thoraco-Abdominal Asynchrony in Children from Motion Point Cloud

In situation of respiratory failure (RF), patients show signs of increase work of breathing leading to involvement of the accessory respiratory muscles and desynchronization between rib cage and abdomen named thoraco-abdominal asynchrony (TAA). The clinical assessment of these signs is a crucial component to get a relevant evaluation of the patient's condition in order to provide the appropriate treatment at the proper time. Proper assessment of these signs requires sufficiently skilled and trained people. However, the human assessment is subjective and is practically impossible to audit. Moreover, there are no standardized reference values of these signs available for use in clinical practice. The purpose of this work is to study the feasibility of visualization and quantification of TAA in patients with RF. In this example, a new non-contact method was developed to visualize surface variation by calculating the 3-dimensional motion of thorax and abdomen surfaces during breathing using a high-fidelity mannequin simulating the thoraco-abdominal asynchrony. An RGB-D sensor was used to visualize the surface variations of the thorax and abdomen simultaneously without placing markers on the body surface. Furthermore, the surface displacement range of movement was calculated in four simulated modes from the normal to the severe TAA mode. Respiratory rates were also calculated based on the analysis of the surface movements.

In a clinical environment, breathing monitoring is an important vital task that is done on a daily basis for different patients' ages. Breathing monitoring mainly comprises an assessment of the chest wall motion and measurement of the physiological parameters such as respiratory rate and tidal volume. While many methods have been developed for physiological parameters assessment, there is still a lack of methods to better assess the chest wall spatial motion during breathing.

Chest wall motion assessment, in clinical practice, is currently based on intermittent human observation and is done through physical examinations. This specific part of the global respiratory assessment isn't quantitative and thus is highly subjective with a high inter-observer variation.

Therefore, an objective assessment of chest wall motion is difficult because there are no medical device reporting quantitative values of the surface displacements to address the severity of patients' disease when the paradoxical motion occurs.

Previous works aimed at quantifying the chest wall movement and detecting asynchrony generally make use of respiratory inductive plethysmography. This contact method requires surrounding the subject body with two belts, one thoracic and one abdominal. However, the application of this technique is still limited by some unresolved issues such as the calibration process and the restrictions of contact with the subject body. Moreover, contact-based methods may create discomfort to the patient and influence his breathing, an effect which is more pronounced in infants.

In this example, there is described a contactless real-time imaging system designed to monitor and observe the most active regions on the thoraco-abdominal surface through a 3D imaging measurement method. The proposed system visualizes deformations of the chest wall during breathing efforts through a 3D imaging measurement method, allowing two parallel pathways for the body wall motion when thoraco-abdominal movements (TAM) occurs. Furthermore, the thorax and abdomen regions were individually analyzed to quantify the thorax-to-abdomen breathing displacement and phase shift. Using an RGB-D sensor, geometric information received from depth was combined with intensity variations in color images in order to estimate a dense 3D motion fields. The proposed system uses a coarse-to-fine multiresolution approach to represent different levels of displacement estimation. The estimation is an optimization problem that is solved based on a primal-dual approximation framework. the displacement distance was calculated for each of the thorax and the abdomen in normal condition and three simulated retraction modes going from the normal breathing mode to the severe mode, using the cloud-to-cloud distance estimation.

Despite the significant progress made in chest wall assessment, there is still a need for methods to visualize and quantify the chest wall motion for a more concrete and precise characterization of respiratory diseases. Indeed, the proposed non-contact methods include breathing waveform estimation, motion data variance in the respiration region and physiological parameters estimation, but they do not include quantitative assessment of the chest wall motion and deformations visualization, without having to use markers attached to the chest wall.

A non-contact system was developed to identify and quantify the motion of the thoraco-abdominal region patterns in patients with TAA. The system uses a single RGB-D camera to estimate a dense and instantaneous 3D motion field corresponding to the motion of the surface due to breathing. To estimate a 3D dense motion field, the proposed system takes advantage from the RGB-D camera's features by using both acquired color and depth data simultaneously, and by exploiting its good spatial and temporal resolution. The approach is thus based on considering these three important factors: spatial resolution, temporal resolution and the use of multiple streams (color and depth data) to get more information about breathing pattern. One objective is to verify that the new non-contact system is efficient and reliable to identify and quantify TAA.

An RGB-D sensor is able to capture three simultaneous streams: Color (RGB), Depth (D) and Infrared Radiation (IR). Multiple RGB-D cameras have been released by Intel and Microsoft over the last few years. However, these devices presently work with a borderline level of acceptance of depth resolution. Most of the new RGB-D cameras provide registered RGB and depth images at a fairly high frame rate (30 Hz), which presents an advantageous setting for the implementation of real-time computer vision algorithms. Kinect sensor has been widely used in many studies due its promising properties. An electronic box which consists of a power supply and a USB extension, is needed to connect the Kinect sensor to a computer, making for a complex and demanding installation. Unlike Kinect cameras, the Asus Xtion is very user friendly, presents a small size and does not require complex installations to be used with a laptop. There is no need for an alimentation cable or a specific USB adapter. Moreover, the Asus Xtion can run well on any computer system, unlike the Kinect sensor which requires a USB 3.0 port, at least for the data transfer between the camera and the computer. Furthermore, the images in the two streams are time-stamped by a common clock. The shutters are not in sync, but the time stamps can be used to match color images to the closest depth images, a significant advantage of the Asus Xtion Pro Live Motion over the Kinect cameras. The main advantage of using the Kinect is the ease of Skeleton detection using the skeleton joints provided in the Kinect SDK (20 joints for the Kinect v1 and 25 joints for the kinectv2). The Asus Xtion Pro Live Motion Sensing Camera therefore has many advantages, and is the camera used in this example.

Optical flow is the computer vision algorithm most widely used to estimate a dense motion. However, optical flow formulation allows the motion estimation only in 2D and not in 3D. Estimating the 3D motion requires more prior information than optical flow. The RGB-D camera provides the additional information that allows for 3D motion estimation, the depth information. Thus, estimating the 3-D motion of points in the scene was considered using both color and depth frames simultaneously.

The aim is to calculate the dense 3D motion field of a scene between two instants of time, t and t+1, using color and depth images provided by the RGB-D sensor. First, a set of color and depth images presenting the same size was considered and acquired at the same time using an RGB-D sensor.

Let M:(Ω∈ [symbol] ²)→ [symbol] ³ denote the motion field, where Ω is the image domain. M is expressed in terms of the optical flow u, v and the range flow w. For any pixel with a nonzero depth value, the bijective relationship Γ: [symbol] ³→ [symbol] ³ between M and V=(u,v,w)^{T} is given by equation (17):

$$M = Γ ( V ) = ( \begin{matrix}\frac{Z}{fx} & 0 & \frac{X}{Z} \\ 0 & \frac{Z}{fy} & \frac{Y}{Z} \\ 0 & 0 & 1\end{matrix} ) ( \begin{matrix}\begin{matrix}u \\ v\end{matrix} \\ w\end{matrix} ) \qquad ( 17 )$$

Equation (17) can be deduced directly from the well-known “pin-hole model”, where fₓ, f_{y} are the focal length values and X, Y, Z the spatial coordinates of the observed point. Following the differential model provided by Horn and Schunk, who provided the first formulation of optical flow, the problem of motion estimation can be formulated as a minimization problem of a certain energy functional. From a general perspective, there are three main points in an optical flow algorithm: 1) the formulation of the energy to be minimized; 2) the discretization scheme; and 3) the solver used to minimize the energy. Hence, the motion field is computed from the resolution of equation (18):

min_{V}{E_{D}(V)+E_{R(V)}} (18)

In equation (18), the sum of the data and regularization terms is minimized over V. The first term E_{D} (V) represents the data term, including both color and depth data, while the second term E_{R}(V) is the regularization term used to smooth the flow field and to constraint the solution space. The resolution of the minimization problem as described in equation (18), can be found in this work, along with the implementation details.

The aim is to regroup motion vectors that have almost the same moving direction (either towards or away from the camera) in order to differentiate between the main surface deformation schemes. These deformations result from air movement into and out of the lungs, which depends upon changes in pressure and volume in the thoracic cavity. Since air is always flowing from an area of high pressure to an area of low pressure, changing the pressure inside the lungs, using the intercostal muscles and the diaphragm determines the direction of airflow and the surface deformation scheme. There are roughly two possible deformations of the 3D surface, either approaching or moving away from the camera. Accordingly, the calculated 3D vector motion fields was divided into a set of two groups, corresponding to inward and outward movements. The Euclidian distance was used, as shown in Equation (19), to assess the similarity between depth motion map vectors' (DMMV) directions. Let [symbol] be the total motion field on the surface S₁. Each 3D vector motion field V(xᵢ₊₁−xᵢ, yᵢ₊₁−yᵢ, zᵢ₊₁−zᵢ)∈ [symbol] is either moving towards (DMMVₒᵤₜ) or away from the camera (DMMVᵢₙ). This is represented by Equations (20) and (21).

dᵢ²=xᵢ²+yᵢ²+zᵢ² (19)

DMMVᵢₙ={Vᵢ∈ [symbol] \d^{t+1}>d^{t},i∈1 . . . N} (20)

DMMVₒᵤₜ={Vᵢ∈ [symbol] \d^{t+1}>d^{t},i∈1 . . . N} (21)

where i indicates a 3D point, (x,y,z) are the spatial coordinate of a 3D point i, V is the motion field of a 3D point i, [symbol] is the total motion field, Nis the number of 3D points over the surface Sₜ and d^{t} is the Euclidian distance from the origin of the coordinate system at frame t. The following mathematical symbol “I” indicates a “such as” condition.

In FIG. 13, the Euclidian distance d^{t} is calculated for all motion vectors at their origins and compared to the distance d^{t+1} at frame t+1. This comparison allows the clustering of the motion vector fields into outward and inward movements. For example, the comparison of the Euclidian distances in V₁, V₂ and V₃ yield to adding V₁ and V₂ to the DMMVₒᵤₜ cluster and V₃ to the DMMVᵢₙ cluster. The surface Sₜ is represented by M 3D point clouds pⱼ, j∈{1 . . . M} at frame t, whose projection is on the surface Sₜ₊₁ at frame t+1 are qⱼ, j∈{1 . . . M}. For every motion vector Vᵢ ∈ [symbol] , the Euclidian distance in the 3D space between vector points and the camera's center are calculated and compared. This comparison allows to determine the motion direction. For V₁,d^{t+1}<d^{t}, V₁ is moving towards the camera (DMMVₒᵤₜ) which correspond to an outward movement. For V₃,d^{t+1}>d^{t} and V₃ is moving away from the camera (DMMVᵢₙ) which corresponds to an inward movement.

Consider M point clouds are provided on each surface and N surfaces. Sᵢₙ^{j} and sₒᵤₜ^{j} can be defined as the set of sub-surfaces of S^{j}, j∈{1 . . . N}, respectively moving inward and outward, as shown in equations (22) and (23). For example, Sᵢₙ¹ is the subsurface of S¹ moving inward. The rest of the surface is set to zero. Indeed, only the points of the surface moving inward in the same direction are kept. Likewise, Sₒᵤₜ¹ is the subsurface of S¹ moving outward.

Sᵢₙ^{j}={pᵢ(xᵢ,yᵢ,zᵢ)∈S^{j}/Vᵢ∈DMMVᵢₙ},j∈{1 . . . N} (22)

Sₒᵤₜ^{j}={pᵢ(xᵢ,yᵢ,zᵢ)∈S^{j}/Vᵢ∈DMMVₒᵤₜ},j∈{1 . . . N} (23)

All measurements are performed on a baby mannequin (SimBaby, Laerdal) designed for medical pediatric simulation with specific anatomical and physiological characteristics. The experiments were done in the simulation center at Sainte-Justine Hospital in Montreal, in conditions similar to a pediatric intensive care unit room.

The experimental environment includes a mannequin used to simulate the retraction, an Asus Xtion RGB-D sensor placed 1 meter over the mannequin and 2 VL53L0X laser-ranging sensors. The VL53L0X sensor is a fully integrated sensing system with an embedded 940 nm infrared VCSEL (vertical-cavity surface-emitting laser) array. VCSELs are known by their narrow and stable emissions when compared to the conventional wide spectrum of LEDs (light-emitting diodes). The VL53L0X distance sensor system uses Time-of-Flight (ToF) technology to accurately measure the distance to a target object. The sensor is independent of the target's color or reflectivity and can report distances of up to 2 m with 1 mm resolution. To detect the invisible laser beam on the mannequin's thoraco-abdominal surfaces, a 940 nm laser detector card was used.

Four situations were recorded, normal breathing mode without any TAA, mild TAA, severe TAA then irregular mode. In normal condition, the thorax and abdomen inflate simultaneously during inspiration and deflate simultaneously during expiration. In TAA, the thorax will deflate while the abdomen inflate, reflecting the high level of negative intra-thoracic pressure during inspiration and during expiration the thorax inflate while the abdomen deflate. In mild condition, thoracic deflate will be less intense compare to the severe condition, thus distance between thorax and abdomen is lower. In irregular mode, the SimBaby will create random cycles with either normal or mild TAA or severe TAA. The mode and the respiratory rate are triggered by a board computer linked to the mannequin. A fixed respiratory rate of 35 breath/minute (BPM) was chosen.

Data over 1 minute were recorded for each mode in this order: normal, mild TAA, severe TAA and irregular mode.

Two sets of experiments were performed. The depth variation of the retraction zones was calculated in the first set of experiments. In this case, the camera is positioned 1 meter above the thoraco-abdominal zone and is pointing downwards. As shown in FIG. 14, the imaging system 1400 is positioned in a vertical or slightly angled position so that variations along the X- and Y-axes are insignificant when tracking the position of a 3D point in the camera coordinate frame. The imaging system 1400 has a camera system including a RGB camera 1406, a first laser range finder 1408 directed to the thorax region 1402 and a second laser range finder 1410 directed to the abdomen region 1404. In the second round of experiments, the viewing angle of the imaging system was validated by calculating the retraction zone depth from different viewing angles. To evaluate the precision of the proposed method, two other sets of data corresponding to the two lasers measures were simultaneously collected.

As shown in FIG. 14, the laser range finders 1408 and 1410 are wrapped around the RDB camera 1406. The first laser range finders 1408 calculates the distance variation in the thoracic region 1402, and the second laser range finders 1410 calculates the distance variation in the abdominal area 1404.

The thoraco-abdominal zone was extracted as described above. This zone includes the areas of interest, whose motion are given by a 3D dense point cloud describing the patient's breathing. The raw data is composed of RGB and depth images. The point cloud (X,Y,Z) is derived from depth images, while the colored point cloud is calculated from both depth and RGB data. As can be expected, the camera system can be used to generate different types of images including, but not limited to, RGB images, depth images, point clouds (X,Y,Z), colorized point clouds (X,Y,Z,R,G,B), segmented ROI images, and scene flow images. In the latter type of image, points of a first color can denote initial positions of 3D points (at frame t) and points of a second color can denote the final positions (at frame t+1).

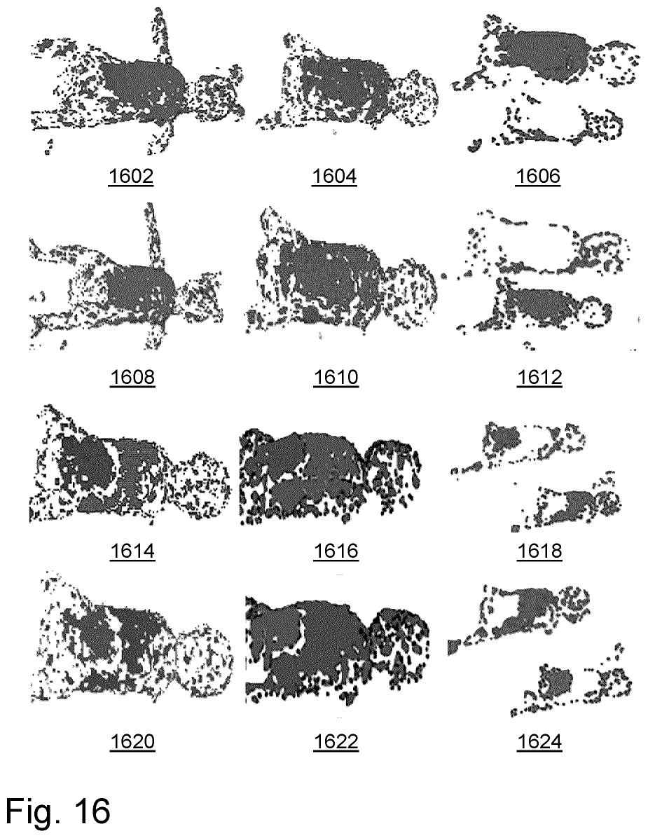

The inspiration movement corresponds to a 3D motion towards the camera, while the expiration is a 3D motion in the opposite direction. In the case of TAA, the two motions occur almost simultaneously at two different zones of the thoraco-abdominal zone. As shown in the succession of 3D images of FIG. 16, the chest and abdomen are moving opposite to each other and this is detected by our extraction technique. Using the proposed method for motion extraction, it is possible to extract two sub-regions according to the inward or forward movement of the point cloud. 3D point clusters moving forward are depicted in red, while 3D point clusters moving backward are colored in red. As shown in FIG. 16, the breathing motion has been simulated using the phantom. Three categories of movements corresponding to the inspiration, expiration and TAA, are clearly seen. During normal inspiration, the lungs are inflated by the expansion and contraction movements of the diaphragm and the ribs that give the thorax its shape. 3D images 1602, 1604 and 1606 represent inspiration motion. Most of the 3D points are colored in red due to the forward movement of both chest and abdomen.

Expiration is a passive movement; the lungs acts like a deflating balloon following by the abdomen. 3D images 1608, 1610 and 1612 represent the expiration motion. Most of the 3D points are colored in blue due to the inward movement of the chest and the abdomen. 3D images 1612 through 1622 represent the paradoxical motion. Since the chest moves in the opposite direction of the abdomen, both red and blue colors can be seen and are more equitably distributed between 3D point clouds. The movements of the rib cage are paradoxical relative to those of the abdomen and to airflow. As shown in 3D images 1614 to 1618 representing inspiration time, the thorax is deflecting thus the region is represented with a blue point cloud and the abdomen point cloud is represented in red. This means that the rib cage is moving inward while the abdomen is moving forward. In 3D images 1618, 1622 and 1624, representing expiration time, the chest region is represented with a red point cloud while the abdomen point cloud is represented in blue. In 3D images 1606, 1612, 1618, and 1624, a translation was applied between the two clusters moving forward and backward in order to visualize them clearly in two different plans.

The set of surfaces S^{j}, j∈{1 . . . N} was considered and daverageᵢₙ^{j} and daverageₒᵤₜ^{j} were defined as the average distances from the camera to the inward Sᵢₙ^{j} and outward Sₒᵤₜ^{j}, moving sub-surfaces, respectively. The distance between a 3D point pᵢ(xᵢ,yᵢ,zᵢ) and the sensor is the euclidien distance, which has been given in equation (19). The cloud-to-sensor distance is defined in this work as the average distance from the camera to the cloud over all 3D points in the cloud. The cloud-to-sensor distance is calculated from the camera to the two sub-surfaces Sᵢₙ^{j} and Sₒᵤₜ^{j}, in order to have the average motion signal for both retraction regions and to estimate the retraction distance on the two regions.

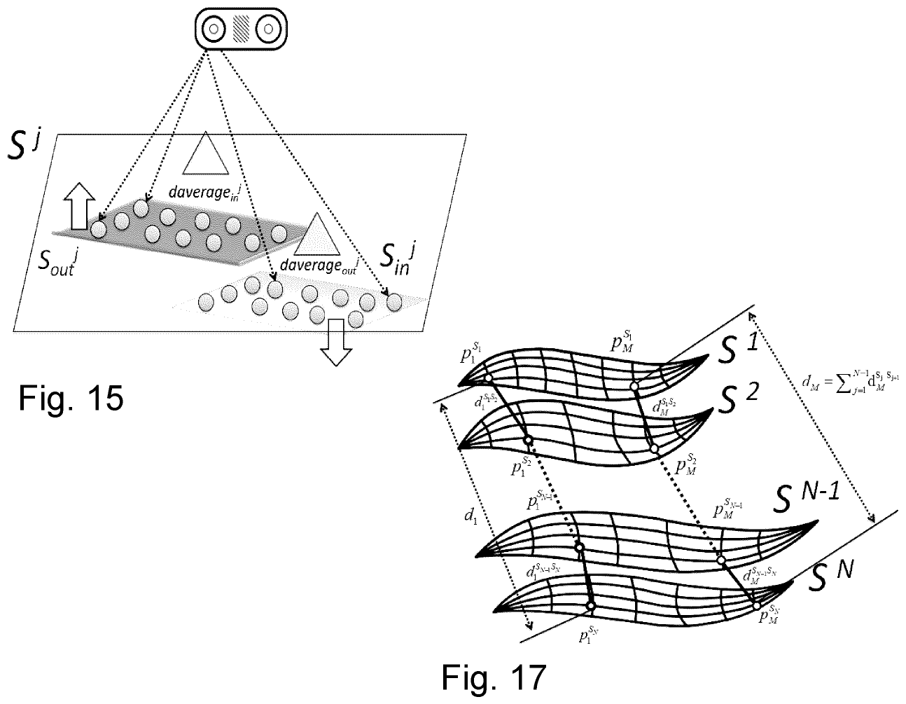

As shown in FIG. 15, the distances daverageᵢₙ^{j} and daverageₒᵤₜ^{j} are calculated for each frame j∈{1 . . . N} between the sensor and the two extracted surfaces Sᵢₙ^{j} and Sₒᵤₜ^{j}, allowing the estimation of chest and abdominal motions.

Tracking 3D points in point clouds data during breathing is complicated in a very acutely-angled position. Displacement variations along the X- and Y-camera axes are more important than in the case when the camera are placed vertically above the thoraco-abdominal zone. For this reason, a method taking displacements along the X- and Y-camera axes was used.

S^{j} and S^{j+1} denote the thoraco-abdominal surfaces at two consecutives frames. Point clouds of surface S^{j+1} are regarded as “target” points p^{S}^{ⱼ₊₁}=(pₓ^{S}^{ⱼ₊₁},p_{y}^{S}^{ⱼ₊₁},p_{z}^{j+1}), whereas the point clouds of the surface S¹ are the original points p^{S}^{ⱼ}i=(pₓ^{S}^{ⱼ},p_{y}^{S}^{ⱼ},p_{z}^{S}^{ⱼ}). The distance between 3D points is calculated using the Euclidian distance in the space [symbol] ³. The aim is to find the corresponding 3D points before and after the surface displacement from S^{j} to S^{j+1}. Consider that M source points are provided in cloud pᵢ^{S}^{ⱼ} on the surface Sⱼ. Points pᵢ^{S}^{ⱼ},i∈{1 . . . M} from S^{j} are projected on S^{j+1} using the normal vector at each source point. The projected points are noted pᵢ^{′Sj+1},i∈{1 . . . M}. To find a corresponding destination point in S^{j+1}, the nearest neighbor is selected in pᵢ^{S}^{ⱼ₊₁}, i∈{1 . . . M}. The displacement distance is then computed for each pair in the cloud using equation (19), where pᵢ^{S}^{ⱼ} represents the “initial” point on the S^{j} surface and pᵢ^{S}^{ⱼ₊₁} is the “target” point on the S^{j+1} surface.

In FIG. 17, the source point p₁^{S}^{ⱼ₊₁} on the surface S^{j} (cloud in the frame j) is projected on the surface S^{j+1} (cloud in frame j+1) using the normal vector in p₁^{S}^{ⱼ}. As can be seen, the nearest neighbors of the projected point p₁^{′Sj+1} are p₂^{S}^{ⱼ₊₁} and p₁^{S}^{ⱼ₊₁}, p₂^{S}^{ⱼ₊₁}, p₃^{S}^{ⱼ₊₁}, and p₄^{S}^{ⱼ₊₁}. Since p₁^{S}^{ⱼ₊₁} is closest point to the projection p₁^{′Sj+1}, it will be selected as the corresponding point of p₁^{S}^{ⱼ}. Finally, the displacement distance d₁^{S}^{ⱼ}^{S}^{ⱼ₊₁} is computed for the pair (p₁^{S}^{ⱼ},p₁^{S}^{ⱼ₊₁}) by calculating ∥p₁^{S}^{ⱼ₊₁}−p₁^{S}^{ⱼ₊₁}∥₂. By iterating the procedure of finding corresponding 3D points between consecutives frames and calculating the distance between initial points and their projections, a vector of distances dᵢ=(dᵢ^{S}^{ᵢ}^{S}^{₂}, dᵢ^{S}^{₂}^{S}^{₃}, dᵢ^{S}^{₃}^{S}^{₄} . . . dᵢ^{S}^{_{N-1}}^{S}^{_{N}}⁾, i∈{1 . . . N} can be obtained. The Δdᵢ distance was calculated by summing the distances between the different projections of the initial 3D point (sum of dᵢ vector components). Δd is the maximum of Δdᵢ over M point clouds (i∈{1 . . . M}).

To summarize, consider that M source points cloud pᵢ^{S}^{₁}, i∈{1 . . . M} are provided over the surface S₁ and N surfaces (S₁, S₂, . . . , S_{N}). The algorithm includes two main steps. First, correspondences between 3D points and their projections on the consecutive surface are found, and then the distance between each 3D point and its projection was calculated. Indeed, the different distances dᵢ^{S}^{ⱼ}^{S}^{ⱼ₊₁}, i∈{1 . . . M} and j∈{1 . . . N} was computed between clouds for each 3D point on the surface Sⱼ and its projection on the surfaces Sⱼ₊₁. The maximal displacement between S₁ and S_{N} is given by equation (24).

$$Δ d = {max}_{i ∈ \{ 1 … M \}} ( {\sum }_{j = 1}^{N - 1} {d}_{i}^{{S}_{j} {S}_{j + 1}} ) \qquad ( 24 )$$

Note that cloud-to-cloud maximal displacement is calculated over the two sub-surfaces Sᵢₙ^{j} and Sₒᵤₜ^{j}. The technique can obtain the direction of the surface motion, estimates the distance of the different 3D point paths after displacement and calculates the maximal path.

In the first experiment, the camera and the two lasers are placed vertically to the thoraco-abdominal zone, which makes variations negligible along the X- and Y-axes. Experiments were performed for normal condition and 3 modes: mild, severe and irregular. 3D point clouds moving in the same direction have been grouped in the same cluster by using the technique presented above. Indeed, the motion extraction technique determines the number of sub-surfaces. In normal respiration, only one region corresponding to inspiration or expiration is extracted. In TAA, two sub-regions, corresponding to the motion of the thorax and the abdomen are extracted. The average distance is calculated relative to each sub-region of 3D point clouds, using the technique also described above.

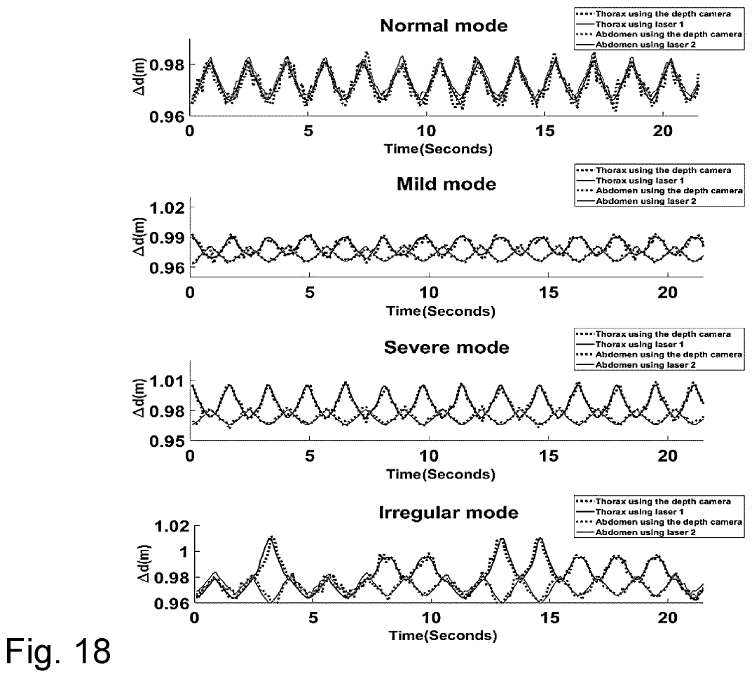

The results obtained from the setup in Figs are illustrated in FIG. 18, which shows the results of the four experiments corresponding to the normal respiration, mild TAA, severe TAA, and irregular mode. It was demonstrated that both techniques (laser and video) are correlated and reliable whatever the conditions. Thoracic and abdominal movements are in-phase with synchronous movements of the two components in normal mode. The signals are showing a characteristic pattern of paradoxical motion with the two components working in opposition in TAA modes. The maximum-to-minimum amplitude between thoracic and abdominal signals represents the retraction difference between the two regions of interest. In the irregular mode, thorax and abdomen are in phase during a normal cycle and in opposition during TAA cycle in random order. Intensity of opposition is different regarding severity of TAA.

The retraction distance can be calculated by averaging the maximum-to-minimum amplitude between the thorax and abdomen respiration signals during a minute of recording, for instance. The respiratory rate can be calculated by simply counting the number of peaks in a minute. However, to improve the accuracy of our method, equation (12) was used, where RR, expressed as the number of respirations per minute, is the respiratory rate, N is the number of peaks during the observation time ΔT (in seconds).

The retraction distance was found to be 1.95±2.4 mm in mild mode, 3.64±4.1 mm in severe mode, and 2.77±1.1 mm in the irregular mode. Results show a very good correlation between the two methods for the 4 modes (>0.985) and a small RMSD of 1.78 in normal mode, 2.83 mm in mild mode, 2.23 mm in severe mode, and 2.34 in irregular mode. In the normal mode, thoracic and abdominal signals are in-phase and hence, Δdₗₐₛₑᵣ and Δd_{camera} are calculated by considering the maximum-to-maximum amplitude between the method (camera) and the reference signal (laser). It was noticed that the amplitude of the abdominal region signal is lower than that at thorax region in both severe and mild modes and is slightly higher in the normal mode. The respiratory rate is 34.75±0.4 BPM in normal mode, 35.19±0.2 BPM in mild mode, 34.8±0.35 BPM in severe mode and 34.66±0.5 BPM in the irregular mode.

The experiments yielded high accuracy and showed significant agreement between the proposed method and the method using laser-ranging sensors when the camera is placed in a vertical position. However, placing the camera in a vertical position above the patient may be problematic when deploying the system in the pediatric intensive care environment. Any occupied space should not cause care interruptions, or present a potential risk for patient safety. Moreover, caregivers need to provide the appropriate services with sufficiently free space around the patient. According to doctors, bed bottom positions are usually the most appropriate to place the camera. In this sub-section, the system's performance was studied when the camera is placed in many positions around the bed, mainly at the bed top (camera #1 and #2) and bottom positions (camera #3 and #4).

The cloud-to-cloud distance metric yields similar findings to those obtained using the camera-to-cloud metric, which confirms the applicability of the proposed system in an intensive care environment. Furthermore, the camera can be placed in both top and bottom positions of the patient's bed. However, placing the camera at the top of the bed yields slightly better results. The slight difference in performance between top and bottom positions is due to the camera depth resolution, which varies with distance from the sensor. Nevertheless, the accuracy in the bottom position is considered acceptable for the calculation of the retraction distance.

This examples presents a new non-contact vision-based method for monitoring acute respiratory failure in a pediatric intensive care environment. The proposed system uses a depth sensor to track the thoracic and abdominal surface motion with high spatial and temporal resolutions. The 3D motion field was computed in each time frame using the collected RGB-D data.

This example relates to assessing retraction signs during the respiratory movement of a patient. Results confirm the accuracy of the proposed method in the estimation of retraction zone distance with a significant agreement compare to a laser distance sensor system. Accuracy is slightly better in bed head position than bottom positions due to the hardware limitation.

### Example 3—Towards a Computer Vision-Based Quantification of Respiration and Chest Wall Deformities During Respiratory Distress in Children

The primary function of the respiratory system is to maintain a normal gas exchange between oxygen (O₂) and carbon dioxide (CO₂) in the lungs. Under normal conditions, O₂ is absorbed into the bloodstream and CO₂ is breathed out. Oxygenated blood travels from the lungs through the pulmonary veins and into the left side of the heart, which pumps the blood to the rest of the body. CO₂ is formed from the metabolism of carbohydrates, fats, and amino acids, in a mechanism known as cellular respiration. CO₂-rich blood returns to the right side of the heart through two large veins. Then the blood is pumped through the pulmonary artery to the lungs, where CO2 is exhaled from the human organism.

Respiratory failure is a critical condition resulting from inadequate gas exchange by the respiratory system, implying that oxygen in the blood becomes dangerously low and/or the level of carbon dioxide in the blood becomes dangerously high. As a result, enough amount of oxygen cannot reach the internal organs (e.g., heart, brain), which may cause serious damage which may lead to death. Acute respiratory distress syndrome (ARDS) is a type of breathing failure resulting from many different disorders that cause fluid to accumulate in the lungs and oxygen concentration in the blood to be very low.

Upper body movement can be a sign that the child suffers from a breathing problem. When children suffer from ARDS, they show signs of increased work of breathing and the involvement of secondary respiratory muscles to keep the concentrations of oxygen and carbon dioxide at normal levels in the organism. Alongside the participation of secondary muscles to get air into the patient lungs, the lack of air pressure causes the skin and soft tissue in the chest wall to sink in. This is called a chest retraction. This disorder is mainly resulting from the weakness of respiratory muscles.

Muscles of breathing include primary muscles, e.g., the diaphragm, intercostals, and secondary muscles. The diaphragm works like a piston to expand the thorax and displace abdominal organs caudad. Intercostal muscles participate in both inspiration and expiration. The thoracic secondary muscles elevate the ribs and facilitate inspiration. The abdominal muscles facilitate expiration. The respiratory muscles can fail for several reasons, as might occur in pneumonia, asma, lung infection by a respiratory virus or even from lung immature development in newborns. As the patient attempts to breathe, the secondary muscles may be excessively over-used to compensate for the mechanics of breathing dysfunction. The workload can lead to respiratory muscle fatigue and then to a cardiopulmonary arrest. Children with deep retractions are treated in the pediatric intensive care unit (PICU) because many of them need mechanical ventilation assistance to breath. The identification of those at risk, and intervening before respiratory failure occurs, is a critically important skill for pediatric clinicians.

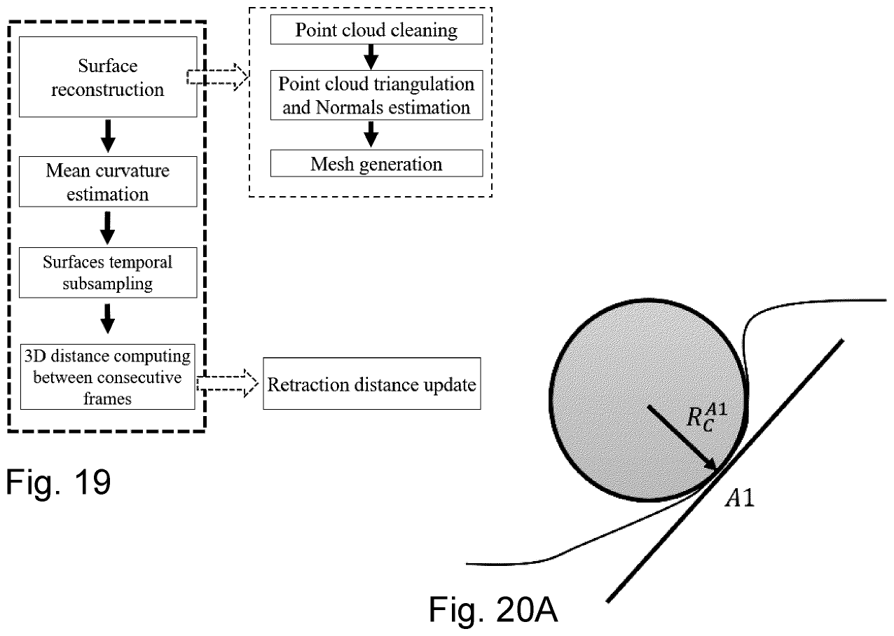

Retraction may occur in several locations of the chest wall. For example, intercostal retractions are observed through the inward movement of the skin between the ribs. Retraction types are shown in FIG. 19. These abnormal patterns can be discernible by an expert's visual inspection, especially in babies and small children whose torsos are softer and may not be fully grown yet. The intensity of work of breathing may be reflected through slight (shallow) or significant (deep) retractions. The severity of retractions increases with the difficulty of breathing. While shallow retractions are barely visible to the naked eye, the deep retractions are detectable through a visual inspection. However, the classification of their gravity (shallow or deep) is highly correlated to the clinician's expertise.

This subjectivity is problematic, especially when healthcare resources such as pediatric experts are limited. Objective assessment of chest wall motion, on the other hand, is difficult because there are no standard medical devices reporting quantitative values of the chest wall retractions to address the severity of patients' disease.

In this example, a depth-based method is proposed to assess chest wall retractions by estimating the inward movement distance of the retracting region against the rest of the chest wall surface. For data recording, the Microsoft Azure RGB-D sensor is used. This sensor is based on the Amplitude Modulated Continuous Wave (AMCW) Time-of-Flight (ToF) technology. The estimated measures are well correlated with transmitted signals of a highly configurable monitor and with a mannequin factory specifications.

As discussed above, an RGB-D camera can be used to detect and quantify the desynchronization between the rib cage and abdomen compartments known as thoraco-abdominal asynchrony (TAA) or “see-saw” breathing, which is another abnormal pattern. In this example, a new method is proposed to quantify the chest wall retractions such as intercostal and substernal retractions. Experiments were conducted using a high-fidelity mannequin on a variety of pediatric volumes and chest wall deformities patterns, which is difficult to experiment simultaneously in real patients. As such, this example presents a method for chest wall deformities assessment, including retractions (intercostal and substernal) and thoracoabdominal asynchrony. This example also provides a fully integrated and straightforward system for respiration assessment. The system is quantifying tidal volume, respiratory rate, minute ventilation and chest wall deformities (retractions and see-saw motion).

The proposed method consists of using a re-topologized triangular mesh derived from a photogrammetric point cloud to compute a mean curvature and extract the top and bottom surfaces, corresponding to the end of inspiration and expiration in a respiratory cycle (or vice versa). The overall description of the method is given in FIG. 19, which includes four main phases: (1) surface reconstruction, (2) mean curvature estimation, (3) surfaces temporal extraction, and finally (4) distance computing. The output distance is used to update the retraction distance over the observed period. In this example, the method is used to calculate retraction distance, and also three main respiratory parameters, i.e., respiratory rate, tidal volume, and the see-saw distance.

An RGB-D sensor can capture three simultaneous streams: Color (RGB), Depth (D) and Infrared Radiation (IR). There have been three RGB-D sensors released by Microsoft over the last few years. While previous two versions of Microsoft's Kinect (Xbox Kinect V1 & V2) were primarily focused on gaming. Subsequently, Microsoft released in March 2020 their new Azure DK version, a fully implemented device targeting additional markets using artificial intelligence (AI) and computer vision applications. Previous commercial depth sensors are working with a borderline level of acceptance of depth resolution. The kit of Azure DK includes an upgraded 1MPixel time-of-flight depth camera working with two mode control (a passive IR mode, plus wide and narrow field-of-view depth modes) capable of 640×576 pixels or 512×512 pixels resolutions at 30 fps, or 1024×1024 pixels resolution at 15 fps. The sensor includes an ultra-HD 12 MPixel RGB camera as well with 3840×2160 pixels at 30 fps (compared to 1920×1080 pixels at 30 fps for its previous version the Kinect V2). Other types of 3D cameras can be used in other embodiments.

First, the surface is scanned, and the point cloud is computed from the scan. Point clouds are sets of 3D points that represent the external surface of a scanned physical object in the 3D space. While this representation is useful for many 3D applications, the point cloud is not sufficient to perform some operations like estimating object curvatures and volumes. The aim of this first stage is to provide a close triangulated mesh to the scanned object. Triangulation is a common method to discretize and generate a surface from point clouds. A triangular mesh has the advantage of creating flat panels between three points. Therefore, a planar triangle mesh can approximate any given surface. The sub-steps are described in FIG. 19. A triangulated mesh is created by means of three main sub-stages: (1) cleaning the point cloud, (2) computing and orienting the normal, (3) mesh generation using the Poisson reconstruction method. The cloud is cleaned, and artefacts are removed using the Statistical Outlier Removal (S.O.R) filter. The minimum spanning tree propagation algorithm was used to compute and compute the normal of each flat panel.

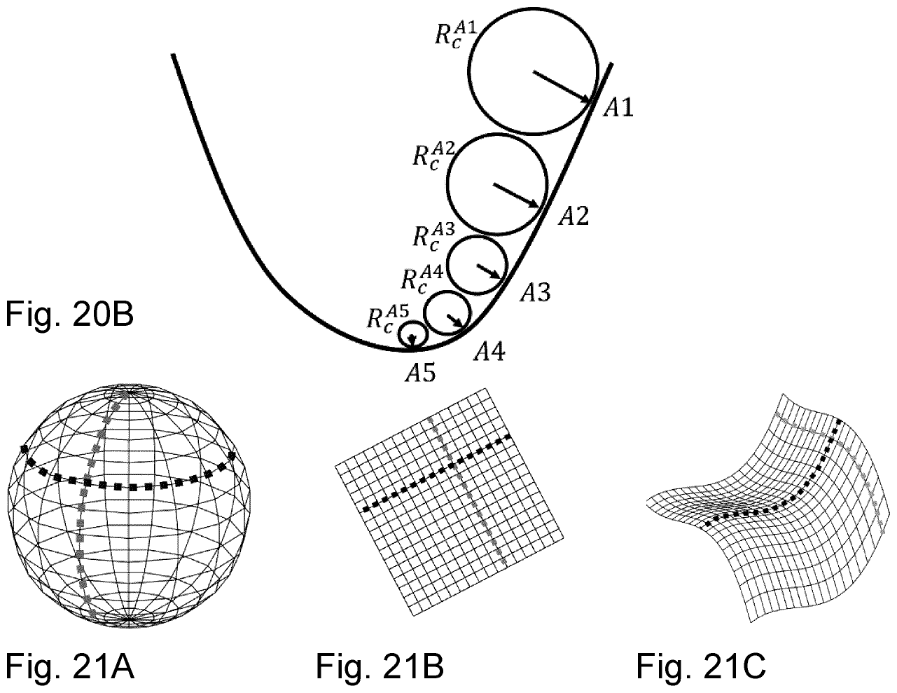

The curvature at any point along a curved contour is given by Equation (25), where R_{c} is the radius of an osculating circle at that corresponding point, as shown in FIG. 20A. This radius is called the radius of curvature and is the curvature length scale. FIG. 20B is showing a curved contour with different points Aᵢ, i∈{1 . . . 5}. It can be seen, through this example, that the smaller is the radius, the highest is the curvature and conversely, the larger is the radius, the smallest is the curvature. The highest value of the contour's curvature is represented in point A5 (smallest radius), while its smallest value is represented in point AI (highest radius). It is also noted that a plan is characterized by a zero curvature as the radius is infinite in this example.

$$κ = \frac{1}{{R}_{c}} \qquad ( 25 )$$

Consider that M source points of cloud pᵢ are provided on a given surface S^{j} (surface in the frame j). The curvature at pᵢ along S^{j} is characterized by the principal curvatures κ₁ⁱ and κ₂ⁱ, which are the maximum and minimum curvatures of surface contours that pass through the point pᵢ. In FIGS. 21A-21C, the principal axes of the surface's curvature are indicated through the dashed lines. There are many types of curvatures definitions. The most known are the Gaussian and Mean curvatures. The Gaussian curvature is expressed as the product of the principal curvatures at every point of the surface, as described in Equation (26). The mean curvature is the mean of principal curvatures passing through the surface's 3D points, as expressed in Equation (27). Depending on the principle curves signs, the curvature can be positive, negative, or equal to zero. FIG. 21A presents a mesh of sphere with positives principle curves (dashed lines). The resulting Gaussian curvature is positive. FIG. 21B shows an example of curvature equal to zero while FIG. 21C shows an example of a shape (saddle-like structure) where the principle curves have different signs, which make the Gaussian curvature negative.

$${K}_{G} = {κ}_{1} \cdot {κ}_{2} \qquad ( 26 )$$

$${K}_{M} = \frac{{κ}_{1} + {κ}_{2}}{2} \qquad ( 27 )$$

Gaussian curvatures are mainly useful on smooth object surfaces. For the sake of simplicity, the mean curvature illustrated un Equation (27) has been chosen as a principle metric to estimate curvature mean values from triangulated meshes.

After surface construction, the region of interest (ROI) is extracted. This step depends on the targeted parameter (e.g., retraction, seesaw distance). It should be noted that a precise segmentation of the thoracic-abdominal region is not obtained by finding its boundaries. Instead, a coarse segmentation is performed by extracting a rectangular cuboid including the region over which the indrawing and chest abnormal pattern occurs. The extraction parameters are saved and reiterated over each frame. In case of the substernal retractions, the extraction is performed at the xiphoid and the subcostal level. In the case of the TAA, the extraction is performed at both thoracic (ROI₁) and abdominal (ROI₂) regions.

The mean curvature is then computed over the extracted region. The aim is to extract the top and bottom surfaces, corresponding to the end of inspiration and expiration in a respiratory cycle (or vice versa). Thus, Equation (28) is applied, to compute the curvature evolution, where Kₙ is the curvature of the region ROIₙ, n∈{1 . . . N}, and N is the number of surfaces over the observed time.

DFₙ=|Kₙ₊₁|−|Kₙ|,n∈{1 . . . N} (28)

SGN(DFₙ,DFₙ₊₁)∈{TRUE,FALSE} (29)

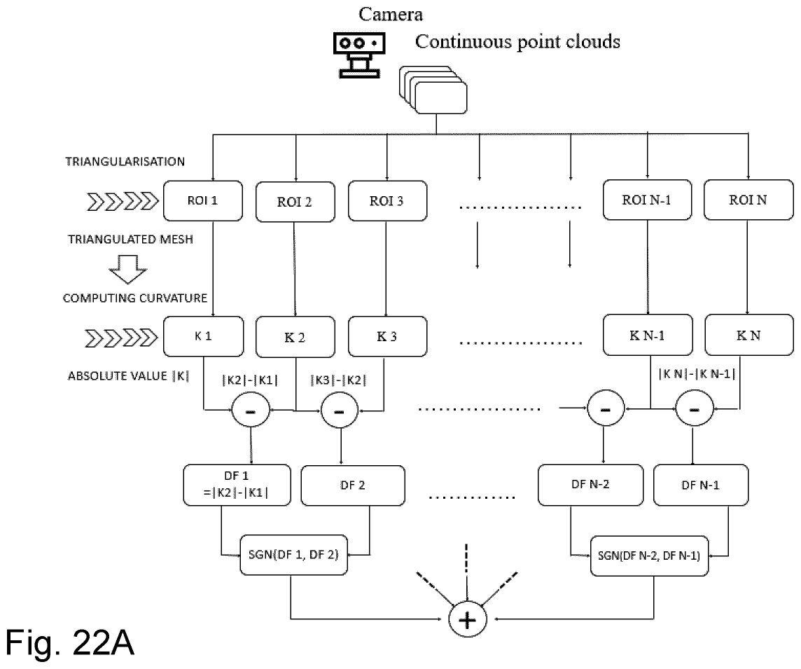

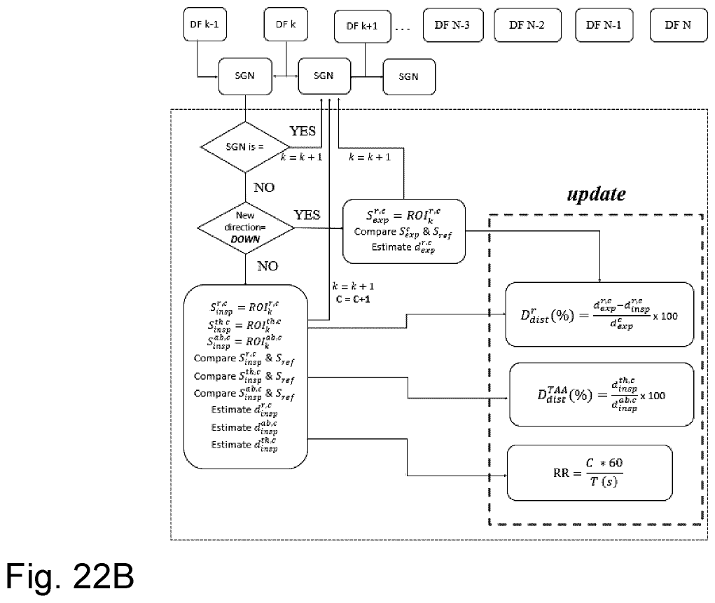

Equation (29) is used to determine whether the consecutive surfaces are moving in the same directions or not. SGN is a Boolean function that returns TRUE if DFₙ and DFₙ₊₁ have the same sign, otherwise it returns FALSE. The program immediately jumps to the next iteration each time SGN (DFₙ, DFₙ₊₁) returns TRUE. Whenever the function SGN (DFₙ, DFₙ₊₁) returns false, the region ROIₙ will be recorded. In this case, if DFₙ<0 and DFₙ₊₁>0, then the direction is changing from downward to upward. Otherwise, if DFₙ>0 and DFₙ₊₁<0, then the movement direction is changing from downward to upward. FIGS. 22A and 22B show the flowchart of the proposed method. A diagrammatic representation of the first steps of the algorithm (from point clouds recording until SGN function computation) is shown in FIG. 22A. The rest of the algorithm, as shown in FIG. 22B, describes the temporal ROI extraction technique. Each time the SGN function returns false, the direction of the surface movement is calculated. At this stage, the surface corresponding to the end of inspiration or the end of expiration is saved and calculate its distance from a reference plan S_{ref} defined by the bed plan.

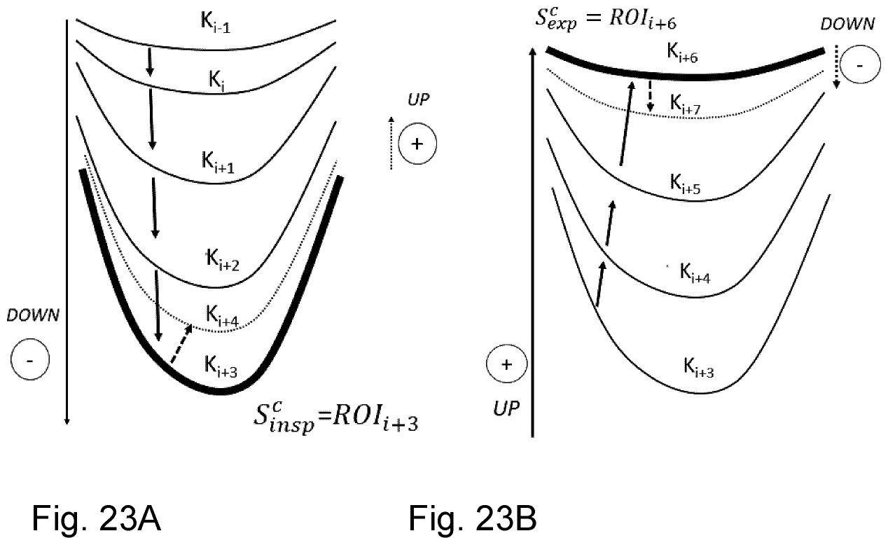

FIGS. 23A-B illustrate an example of the surface extraction technique using the direction changes of the DF, variable. In this example, the sign of DF₁=|Kⱼ₊₁|−|Kⱼ|, j∈{i . . . i+10} is first computed, where i is any given frame number. Results for the next frames are as follows: DFᵢ₊₁>0, DFᵢ₊₂>0, DFᵢ₊₃<0, DFᵢ₊₄<0, DFᵢ₊₅<0, DFᵢ₊₆>0, DFᵢ₊₇>0, DFᵢ₊₈>0, DFᵢ₊₉>0 and DFᵢ₊₁₀<0. The function SGN will return false when detecting a sign change between the input consecutive DF₁ parameters such as in SGN(DFᵢ₊₂,DFᵢ₊₃), SGN(DFᵢ₊₅,DFᵢ₊₆) and SGN(DFᵢ₊₉,DFᵢ₊₁₀). Consequently, only regions with frame number i+3, i+6 and i+10 (second input parameter of the SGN function returning false value) will be extracted. If the direction is changing to upward, then the extracted surface corresponds to the end of an inspiration such as in ROIᵢ₊₃ and ROIᵢ₊₁₁. Otherwise, the surface corresponds to the end of inspiration such as in ROIᵢ₊₆. As such, one can understand that a change of direction is mainly used to extract two surfaces from many 3D images. These two surfaces are those corresponding to the end of inspiration and end of expiration. These 2 surfaces are used to calculate the distance between thorax and abdomen in TAA and the retraction distance in case of other retractions such when accessory muscles are activated. For the rest of this example, the notation of ROIₖ (ROI at frame k) which will be replaced by ROIₖ^{r,c} (retraction ROI at frame k and cycle c) or ROIₖ^{ab,c}/ROIₖ^{th,c} and (abdominal/thoracic ROI at frame k and cycle c).

Respiratory rate (RR), tidal volume (Vt) are estimated in the first phase of the proposed method (surface reconstruction) schematized in FIG. 19. Point clouds, recorded from the 3D cameras, are used to reconstruct a 3D surface of the patient's trunk using the Poisson method or other equivalent method. Poisson surface reconstruction allows to find the best-fitting surface to a dense point cloud. The density can be improved using two depth cameras providing a high spatial coverage of body regions involved in the respiration (top surface and its lateral sides). The method relies on the octree 3D structure representation. Based on a hierarchical tree structure, an octree partitions the 3-D space. Starting from a root node in the form of single large cube, the octree is recursively subdivided into eight equal sized sub-cubes. This subdivision process continues until the regions are empty. The volume is computed for each frame by multiplying the number of octrees by the unit size. Finally, tidal volume and respiratory rate are computed by analyzing the changes in the computed volume-time curve. Equations (12) and (13) have been used to compute RR in BPM and Vt in mL, respectively, where N is the number of peaks of the volume-time curve during the observation time ΔT (in seconds) and tvᵢ is the tidal volume of the cycle i (maximum-to-minimum amplitude difference of the volume-time curve).

For the rest of the steps, only the end-of-inspiration and end-of-expiration surfaces are extracted using the temporal subsampling algorithm described in the next paragraph and in FIGS. 9 and 10.

The ROI is called ROIₖ^{r,c}, where k is the frame number, r stands for retraction and c corresponds to the respiratory cycle. If the direction is changing to upward, the region ROIₖ^{r,c} can be saved as Sₑₓₚ^{r,c} where exp is indicating the end of the expiratory phase. The distance dₑₓₚ^{r,c} between Sₑₓₚ^{r,c} and S_{ref} is then computed and saved to calculate D_{dist}^{r}. If the direction is changing to downward, the region ROIₖ^{r,c} can be saved as Sᵢₙₛₚ^{r,c} where insp is indicating the end of the inspiratory phase. In this case, distance dᵢₙₛₚ^{r,c} between Sᵢₙₛₚ^{r,c} and S_{ref} is computed and used immediately with the previously saved distance dₑₓₚ^{r,c} (for the same cycle C) to compute D_{dist}^{r} using Equation (30). The program will increment the variable C by 1, which corresponds to a new respiratory cycle and then jump to the next iteration (k=k+1).

$${D}_{dist}^{r} ( \% ) = \frac{{d}_{exp}^{r , c} - {d}_{insp}^{r , c}}{{d}_{exp}^{r , c}} * 100 \qquad ( 30 )$$

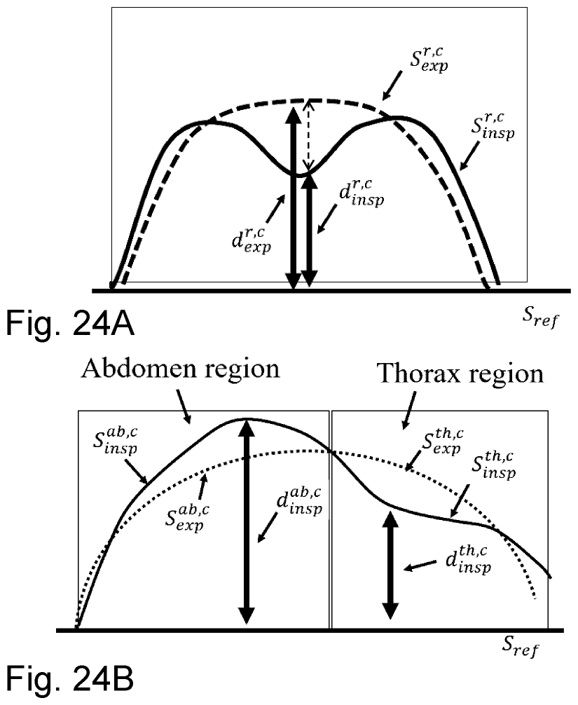

FIGS. 24A-24B show a one-dimensional graphical illustration of both end of inspiration (solid lines) and end of expiration (dashed lines).

In the case of TAA pattern, two ROI were extracted ROIₖ^{th,c} and ROIₖ^{ab,c}, which respectively correspond to the thorax (th) and abdomen (ab) regions at cycle C and frame k. Only inspiratory surface will be saved in Sᵢₙₛₚ^{th,c} and Sᵢₙₛₚ^{ab,c} respectively. Expiratory surfaces are not used to estimate the variation percentage between the thorax and the abdomen. The distances dᵢₙₛₚ^{th,c} (between Sᵢₙₛₚ^{th,c} and S_{ref}), dᵢₙₛₚ^{ab,c} (between Sᵢₙₛₚ^{ab,c} and S_{ref}) are computed and used in Equation (31), which shows the relative variation between the thorax and abdomen regions. Finally, the program will increment the variable C by 1 (new cycle) and then jump to the next iteration (k=k+1).

$${D}_{dist}^{TAA} ( \% ) = \frac{{d}_{exp}^{th , c}}{{d}_{exp}^{ab , c}} * 100 \qquad ( 31 )$$

FIG. 24A shows a one-dimensional graphical illustration of the used technique to estimate the relative variation between the two compartments of the thoraco-abdominal region. The ratio of expansion of the thorax and abdomen regions compared to a fixed reference plan in this example. For retractions which are due to the activation of the accessory muscles to meet ventilation demands (i.e., due to primary muscles workload), one can see that muscles between the ribs pull inward at the end of inspiration too as illustrated in FIG. 24A. However, both surfaces of end of inspiration and end expiration are used to calculate the retraction distance. The system estimates the respiratory rate over the observed period ΔT using Equation (32), where c is the cycle number.

$$R R ( BPM ) = \frac{c}{Δ T} * 60 \qquad ( 32 )$$

The experiments have been conducted in the simulation center of Sainte-Justine Hospital in Montreal. All simulations have been performed using the new SimBaby IRIS, designed according to neonatal anatomical and physiological characteristics. The wireless SimBaby present many features such as head movement, reactive eyes, pulses/sounds producing, liver palpation, normal/abnormal breathing modeling, etc. . . . . The main features used in this work are the spontaneous breathing simulation with variable respiratory rates, breathing complication (Pneumothorax) and chest wall abnormal patterns simulation (Normal-Seesaw-Subcostal Retraction). These features can be triggered using a highly configurable monitor. The head of bed is placed at a 30-degree angle. This position is used for patients who have respiratory problems, and with intubated patients. Two computers are used for data recording. Three set of experiments have been conducted.

In the first experiment, we simulate the breathing activity using both constant and variable breathing rates. Normal spontaneous breathing patients have normal rates, while critically ill patients may have variable rates of respiration. Moreover, we compare the volume estimation using one and two Kinect cameras. It was shown that two Kinect V2 camera can be used to calculate the tidal volume. The system has been validated using a mechanical ventilator, the gold standard in PICU. We showed that the use of two cameras allows to cover the top of the thoraco-abdominal region, as well as its lateral side. Moreover, merging clouds recorded from two different view angles allows to increase the density of the final point cloud, which enhance the quality of the reconstruction. Since the Kinect Azure DK offers a better resolution of the depth (1MP) and high-density point cloud, we make the hypothesis that a single high-resolution Kinect Azure camera may be sufficient to estimate the tidal volume.

The aim of the first experiment is to compare the single and dual camera approaches, in tidal volume estimation. We remind that the dual-camera system has been validated using a mechanical ventilator in the PICU, on both mannequin and two intubated patients.

The proposed system is a very promising support tool intended to assist caregivers in respiration assessment in an intensive care environment. It is envisaged to merge Examples 1, 2 and 3 to one another so as to provide methods and systems being able to monitor respiratory rate, tidal volume measurements as well as detecting retraction signs during the respiratory movement of the patient.

As can be understood, the examples described above and illustrated are intended to be exemplary only. For instance, the method(s) and system(s) described herein can be applied to assess the solicitation of secondary muscles such as the sternocleidomastoid, the scalene muscles, and the intercostal muscles, in the respiratory movement of the patient. This can be assessed by evaluating the motion of the region around the clavicle, the neck, and/or the rib cage. In a distressed respiration, these muscles are solicited and therefore the region around the clavicle, below the neck and/or the region between the ribs will sink. The presence of motion and the quantification of that secondary respiratory motion can indicate and quantify respiratory distress as well. The scope is indicated by the appended claims.

1. A method of assessing severity of a respiratory distress of a patient, the method comprising:
using a three dimensional (3D) camera, generating at least a 3D image encompassing at least a thoraco-abdominal region of said patient at a given moment in time; and
using a computer,
accessing said 3D image;
identifying first coordinates indicating coordinates of at least a first point of said thoraco-abdominal region of said patient in said 3D image;
identifying second coordinates indicating coordinates of at least a second point of said thoraco-abdominal region of said patient in said 3D image;
determining a distance based on said first and second coordinates;
comparing said distance with a threshold; and
generating a signal based on said comparison, said signal being indicative of a degree of severity of said respiratory distress of said patient.

2. The method of claim 1 wherein said thoraco-abdominal region has at least a thorax region and an abdominal region, said first point being associated with said thorax region of said patient in said 3D image, said second point being associated with said abdominal region of said patient, and said distance corresponding to a thoraco-abdominal distance indicating a distance between said thorax region and said abdominal region of said patient.

3. The method of claim 1 wherein said thoraco-abdominal region has at least a secondary respiratory muscle and an anatomical landmark, said first point being associated with said secondary respiratory muscle of said patient in said 3D image, and said second point being associated with said anatomical landmark of said patient in said 3D image.

4. The method of claim 3 wherein said secondary respiratory muscle is selected among a group consisting of: a sternocleidomastoid muscle, a scalene muscle, and an intercostal muscle.

5. The method of claim 3 wherein said anatomical landmark is selected among a group consisting of: a region around a clavicle of said patient, a region below a neck of said patient and a region between ribs of said patient.

6. The method of claim 1 further comprising generating an alert when said distance exceeds said threshold.

7. The method of claim 1 wherein said moment in time corresponds to at least one of an end of an inspiration and an end of an expiration of said patient.

8. The method of claim 1 further comprising repeating said method a given number of times thereby monitoring said distance over time.

9. The method of claim 8 further comprising displaying said monitored distance on a display screen.

10. The method of claim 1 wherein said 3D image is provided in the form of a cloud of points.

11. A system for assessing severity of a respiratory distress of a patient, the system comprising:
a three dimensional (3D) camera generating at least a 3D image encompassing at least a thoraco-abdominal region of said patient at a given moment in time; and
a computer being communicatively coupled to said 3D camera, said computer having a processor and a memory having stored thereon instructions that when executed by said processor perform the steps of:
accessing said 3D image;
identifying first coordinates indicating coordinates of at least a first point of said thoraco-abdominal region of said patient in said 3D image;
identifying second coordinates indicating coordinates of at least a second point of said thoraco-abdominal region of said patient in said 3D image;
determining a distance based on said first and second coordinates;
comparing said distance with a threshold; and
generating a signal based on said comparison, said signal being indicative of a degree of severity of said respiratory distress of said patient.

12. The system of claim 11 wherein said thoraco-abdominal region has at least a thorax region and an abdominal region, said first point being associated with said thorax region of said patient in said 3D image, said second point being associated with said abdominal region of said patient, and said distance corresponding to a thoraco-abdominal distance storable on said memory.

13. The system of claim 11 wherein said thoraco-abdominal region has at least a secondary respiratory muscle and an anatomical landmark, said first point being associated with said secondary respiratory muscle of said patient in said 3D image, and said second point being associated with said anatomical landmark of said patient in said 3D image.

14. The system of claim 13 wherein said secondary respiratory muscle is selected among a group consisting of: a sternocleidomastoid muscle, a scalene muscle, and an intercostal muscle.

15. The system of claim 13 wherein said anatomical landmark is selected among a group consisting of: a region around a clavicle of said patient, a region below a neck of said patient and a region between ribs of said patient.

16. The system of claim 11 further comprising an indicator generating an alert when said distance exceeds said threshold.

17. The system of claim 11 wherein said moment in time corresponds to at least one of an end of an inspiration and an end of an expiration of said patient.

18. The system of claim 11 further comprising repeating said 3D camera generates a plurality of 3D images as said patient breathes, said instructions being performed for at least some of said 3D images thereby monitoring said distance over time.

19. The system of claim 18 further comprising a display screen displaying said monitored distance.

20. A method of assessing severity of a respiratory distress of a patient, the method comprising:
using a three dimensional (3D) camera, generating a plurality of 3D images encompassing at least a thoraco-abdominal region of said patient at a plurality of moments in time; and
using a computer,
accessing said plurality of 3D images;
identifying a plurality of thoraco-abdominal coordinates indicating coordinates of at least a point of said thoraco-abdominal region of said patient in said plurality of 3D images;
determining a direction of movement of said thoraco-abdominal region across said moments in time based on said identified thoraco-abdominal coordinates;
upon determining that said direction of movement switches from a first direction of movement to a second direction of movement opposite to said first direction of movement, identifying at least one of a first 3D image of said plurality of 3D images corresponding to an end of an inspiration of said patient and a second 3D image of said plurality of 3D images corresponding to an end of an expiration of said patient; and
generating a signal based on at least one of said first and second 3D images, said signal being indicative of a degree of severity of said respiratory distress of said patient.

21.-55. (canceled)
